(19) United States
(12) Patent Application Publication
(10) Pub. No.: US 2010/0081630 A1
(43) Pub. Date: Apr. 1, 2010

(54) Heparin Compositions and Selectin Inhibition

(75) Inventors: Ajit Varki, Del Mar, CA (US); Jennifer L. Stevenson, Santa Clarita, CA (US)

Correspondence Address: MEDLEN & CARROLL, LLP, 101 HOWARD STREET, SUITE 350, SAN FRANCISCO, CA, 94105, US

(73) Assignee: University of California

(21) Appl. No.: 12/630,106

(22) Filed: Dec. 3, 2009

Related U.S. Application Data
(62) Division of application No. 11/491,388, filed on Jul. 21, 2006.
(60) Provisional application No. 60/701,893, filed on Jul. 22, 2005.

(51) Int. Cl. A61K 31/727; C12Q 1/48

(52) U.S. Cl. 514/56; 435/15

(57) ABSTRACT

The disclosure provides in vitro and in vivo methods for identifying Heparins and Heparinoids that modulate the activity of selectins. The disclosure also provides Heparins and Heparinoids that modulate the activity of selectins. The identification and isolation of these heparin formulations has the potential to mediate a wide variety of pathologies mediated by P- and/or L-selectin, including hematogenous metastasis, diseases associated with inflammation (e.g., asthma, arthritis, allergic dermatitis), ischemia-reperfusion injury, or other pathologies such as sickle cell anemia. Selectin inhibition can be achieved at plasma concentrations lower than those that cause excessive anticoagulation or unwanted bleeding in a human subject.

### CROSS REFERENCE TO RELATED APPLICATIONS

This application claims priority to U.S. Provisional Application Ser. No. 60/701,893 filed Jul. 22, 2005, the disclosure of which is incorporated herein by reference.

### STATEMENTS REGARDING FEDERALLY SPONSORED RESEARCH

The invention was funded in part by Grant No. R01CA38701 awarded by the National Institutes of Health. The government may have certain rights in the invention.

### TECHNICAL FIELD

The disclosure relates generally to molecular biology and, more specifically, to methods of identifying and/or isolating heparin variants that block the binding activity of L-selectin and/or P-selectin and attenuate selectin-mediated metastasis or other selectin-mediated diseases or disorders.

### BACKGROUND

P- and L-selectin are C-type lectins that recognize sialylated, fucosylated, sulfated ligands. P-selectin is stored within resting platelets and endothelial cells, and translocates to the cell surface upon activation. L-selectin is constitutively expressed on most leukocyte types and mediates their interactions with endothelial ligands. Both selectins promote the initial tethering of leukocytes during extravasation at sites of inflammation. P-selectin also plays a role in hemostasis. Endogenous ligands for P- and L-selectin (such as PSGL-1) are expressed on leukocytes and endothelial cells (for general reviews on selectins and their ligands (see, e.g., Varki A., Proc Natl Acad Sci USA (1994) 91:7390-7; Ley et al., J Immunol (1995) 155:525-8; Kansas G S, Blood (1996) 88:3259-87; McEver et al., J Clin Invest (1997) 100:485-92; Lowe J B. Kidney Int (1997) 51:1418-26; Rosen S D. Annu Rev Immunol (2004) 22:129-56).

P- and L-selectin also have pathological roles in many diseases involving inflammation and reperfusion (Bevilacqua et al., Annu Rev Med (1994) 45:361-78; Lowe et al., J Clin Invest (1997) 99:822-6; Ley K., Trends Mol Med (2003) 9:263-8), as well as in carcinoma metastasis. Many tumor cells express selectin ligands, and an inverse relationship between tumor selectin ligand expression and survival has been reported (Varki N M, Varki A. Semin Thromb Hemost (2002) 28:53-66). Syngenic and allogenic mouse models have demonstrated that metastasis of selectin ligand-positive adenocarcinomas to the lungs is P- and L-selectin-dependent (Kim et al., Proc Natl Acad Sci USA (1998) 95:9325-30; Friederichs et al., Cancer Res (2000) 60:6714-22; Borsig et al., Proc Natl Acad Sci USA (2001) 98:3352-7; Borsig et al., Proc Natl Acad Sci USA (2002) 99:2193-8; Ludwig et al., Cancer Res 2004; 64:2743-50).

Many classic studies documented an inhibitory effect of unfractionated heparin (UFH) in animal models of cancer metastasis (Zacharski et al., Thromb Haemost (1998) 80:10-23; Engelberg H. Cancer (1999) 85:257-72; Hejna et al., J Nat Cancer Inst (1999) 91:22-36; Smorenburg et al., Pharmacol Rev (2001) 53:93-105), and retrospective analyses indicated that heparin may have similar effects in human cancer (Kakkar et al., Int J Oncol (1995) 6:885-8; Hettiarachchi et al., Thromb Haemost (1999) 82:947-52; Ornstein et al., Haemostasis (1999) 29 Suppl. 1:48-60; Smorenburg et al., Thromb Haemost (1999) 82:1600-4; and Zacharski et al., Semin Thromb Hemost (2000) 26 Suppl. 1:69-77). A large body of literature also discusses the well-documented relationships of cancer and venous thrombosis, and the inhibition of metastasis via blocking fluid-phase coagulation, either with heparin or hirudin. However, human trials using Vitamin K antagonists as an alternate mode of anticoagulation showed no effect on survival in most carcinomas. Thus, it should not be assumed that heparin efficacy in metastasis is based primarily on its anticoagulant activity.

Unfractionated heparin has been in clinical use based on its ability to inhibit fluid phase coagulation by enhancing antithrombin inactivation of Factors IIa and Xa. However, UFH is a natural product containing a complex polydisperse mixture of highly sulfated glycosaminoglycan chains ranging from 5000 to 30000 daltons, only some of which actually bind antithrombin. Early studies showed that P-selectin could bind to immobilized heparin (Skinner et al., Biochem Biophys Res Commun (1989) 164:1373-9). It has been shown that various heparins and heparinoids could inhibit binding of both P- and L-selectin to their natural ligands (Nelson et al., Blood (1993) 82:3253-8; Norgard-Sumnicht et al., Science (1993) 261:480-3; Koenig et al., J Clin Invest (1998) 101:877-89; Ma et al., J Immunol (2000) 165:558-65; Xie et al., J Biol Chem (2000) 275:30718-1).

The identification of pharmaceutical grade heparin and heparinoid preparations useful for inhibiting the binding of L-selectin and P-selectin to ligands present on cells in humans is desirable. Such preparations can be further refined to identify those that not only mediate L-selectin and P-selectin activity, but do so without producing undesirable side effects in a subject.

### SUMMARY

Provided herein are methods for identifying various heparins/heparinoids (hereafter collectively referred to as heparins) for their ability to inhibit the activity of P/L-selectin. Also provided are a subset of heparins that inhibit metastasis in two different tumor models at clinically-relevant doses. Additionally, the invention identifies structural differences between the low molecular weight heparins (LMWHs) in view of their differential selectin-inhibition activity and addresses the relative roles of anticoagulation and selectin inhibition in attenuating metastasis.

In one embodiment, a method for screening a composition for inhibition of selectin activity is provided. The method may include providing a heparin preparation including a plurality of heparin molecules. Generally the preparation is obtained from an FDA-approved heparin lot. Also included in the method are one or more selectins selected from the group consisting of L-selectin and P-selectin; a ligand for one or more of the selectins; and heparin. The method further includes contacting the above-identified items, simultaneously or consecutively, under conditions suitable for selectin binding to a selectin ligand and detecting a reduced level of binding of the one or more selectins to a ligand in the presence of the heparin preparation compared to in the absence of the heparin preparation.

A reduced level of binding between a selectin and a selectin ligand may be detected in a concentration of the heparin preparation that is lower than the concentration of heparin that produces one or more activities selected from the group consisting of anticoagulant activity in vivo and undesirable bleeding in vivo. Further, the concentration of the heparin preparation may not reduce the level of binding of E-selectin to an E-selectin ligand. Moreover, the concentration of heparin that produces the reduced level of binding of the one or more selectins to the ligand may be from 2-fold to 50-fold lower than the concentration of heparin that produces excessive anticoagulant activity in vivo. In some embodiments, it is possible to identify heparins that selectively inhibit selectins. Such heparins will typically lack other heparin activities (e.g., angiogenesis inhibition, heparanase inhibition, cytokine binding and the like). Furthermore, it is possible to identify heparin fractions that only have anticoagulant activity but lack other activities.

Heparin preparations identified by methods provided herein may be used as a therapeutic for L-selectin or P-selectin related pathologies.

The invention also provides a method for screening a composition for inhibition of selectin activity. The method may include providing a heparin preparation including a plurality of heparin molecules. Generally, the preparation is obtained from an FDA-approved heparin lot. Also included in the method are one or more selectins selected from the group consisting of L-selectin and P-selectin; a ligand for one or more selectins selected from the group consisting of L-selectin and P-selectin; and heparin. The method may further include fractionating the heparin preparation and isolating a plurality of fractions comprising heparin molecules, wherein the fractions are isolated based on the size of the heparin molecules in the fraction. The method further includes contacting each fraction with the ligand and selectin, simultaneously or consecutively, under conditions suitable for selectin binding to a selectin ligand and detecting a reduced level of binding of the one or more selectins to a ligand in the presence of the fraction(s) and identifying the fraction(s) that reduce the level of binding of the one or more selectins to the ligand in the presence of the fraction compared to in the absence of the fraction.

The invention also provides a method to identify a heparin fraction as a therapeutic for a L-selectin and/or P-selectin related pathology.

The invention also provides a heparin fraction identified by a method disclosed herein.

The invention provides an article of manufacture including packaging material. Contained within the packaging material may be a heparin preparation identified by a method provided herein. The packaging material may include a label or package insert indicating that the heparin preparation inhibits the activity of a selectin and can be used for inhibiting hematogenous metastases in a subject. The heparin preparation may include a low molecular weight heparin (LMWH) preparation. Exemplary preparations include Tinzaparin (TINZ). In another embodiment, an article of manufacture including packaging material is provided. Contained within the packaging material may be a heparin fraction identified by a method provided herein. The packaging material may include a label or package insert indicating that the heparin fraction inhibits the activity of a selectin and can be used for inhibiting hematogenous metastases in a subject. In one embodiment, the article of manufacture comprises a heparin fraction useful for a specific heparin activity based upon use of the methods of the invention. For example, the article of manufacture comprising a heparin fraction can comprise a label or package insert indicating that the heparin fraction is useful for inhibiting the activity of a selectin and can be used for inhibiting P- and or L-selectin-mediated diseases in a subject.

The invention also provides a method for preventing or treating a cell proliferation disorder in a subject. The method may include administering to the subject an effective amount of a specific inhibitor of selectin activity, in a pharmaceutically acceptable carrier. Generally the inhibitor will be a heparin preparation or a heparin fraction.

The invention provides a method for preventing or inhibiting metastasis in a subject. The method includes administering to the subject an effective amount of a specific inhibitor of selectin activity, in a pharmaceutically acceptable carrier. Generally the inhibitor is a heparin preparation or a heparin fraction.

The details of one or more embodiments of the disclosure are set forth in the accompanying drawings and the description below. Other features, objects, and advantages will be apparent from the description and drawings, and from the claims.

### BRIEF DESCRIPTION OF DRAWINGS

FIG. 1 shows that clinically utilized heparin preparations show marked differences in their ability to inhibit P- and L-selectin binding to carcinoma ligands. Binding of human colon carcinoma cells to immobilized selectin chimeras was tested in the presence of a range of concentrations of different heparins. Control binding was based on measurements in the presence of buffer alone and background values were measured in 2.5 mM EDTA. Each heparin concentration was tested in triplicate, and the presented data is representative of results from multiple experiments.

FIG. 2 shows that therapeutic ranges of anti-Xa units can be achieved with a single heparin dose. Anti-Xa levels were measured in plasma from multiple mice, 30 min after each mouse received a single “1×” or “3×” subcutaneous dose of various heparins. Each open circle represents one mouse and horizontal bars represent mean values.

FIG. 3 depicts inhibition of metastasis of colon carcinoma cells is achieved at clinically-tolerable levels of UFH and TINZ, with FOND (a synthetic pentasaccharide) having no effect. Mice were injected subcutaneously with “1×” heparin (A) or “3×” heparin (B) (or PBS as a control), and 30 minutes later were injected intravenously with MC38GFP cells. After 27 days, mice were euthanized, and metastasis was evaluated by quantifying the fluorescence of lung homogenate. Open circles represent each mouse and horizontal bars represent the mean values. P-values were determined by a Student's T-test, assuming two-tailed, unequal distribution.

FIG. 4 depicts heparins with selectin-inhibitory activity that inhibit metastasis of melanoma cells. Mice were injected subcutaneously with “1×” heparin (A) or “3×” heparin (B) (or PBS as a control), and 30 minutes later were injected intravenously with B16F1 cells. After 17 days, mice were euthanized, lungs perfused with formalin through the trachea and then allowed to fix in formalin for a minimum of 24 hours. Metastasis was quantified by measuring lung weight, which correlated well with the physical appearance of the lungs, documented by photography (representative pictures are shown below quantification). Open circles each represent one mouse and horizontal bars represent mean lung weights. P-values were determined by a Student's T-test, assuming two-tailed, unequal distribution.

FIG. 5 depicts selectin inhibition by TINZ is mediated mainly by high molecular weight fragments with relatively lower anti-Xa activity. (A). Aliquots of the five heparins (UFH, the three LMWHs, and FOND) were run on an HPLC size exclusion system and their size profiles evaluated by tracking absorbance at 206 nm (the relevant part of the chromatogram shown is from t=17.5 to 33.3 minutes). The open arrow marks the elution of the synthetic pentasaccharide FOND. (B) An aliquot of TINZ was run on the same HPLC system as in FIG. 5, and 0.5-minute fractions were collected post UV detector. The total amount (ug) of uronic acid in each fraction was quantified using a carbazole assay. The ability of each fraction to inhibit binding of P-selectin to sLe^{x} was determined, with appropriate dilutions so that all readings were in the linear range (˜30-70% inhibition). One inhibitory unit is arbitrarily defined as 1% inhibition of P-selectin binding. The total number of anti-Xa units in each fraction was also determined in the linear range of that assay (if no activity was detected, the minimum detection limit of the assay was used). Total inhibitory units and total anti-Xa units were normalized to total uronic acid content. If no uronic acid was detected in a sample, the minimum detection limit of the assay was used for the calculation. The hatched box at the top of the graph designates fractions 28-32, which contain high P-selectin inhibitory activity, and minimal anti-Xa activity, when normalized to uronic acid content.

FIG. 6 provides a brief description of possible mechanisms of selectin-inhibitory activity and higher molecular weight heparin fractions. This description is exemplary and in no way limits the disclosed methods and compositions to the described mechanisms. P-selectin (presented by either activated platelets or endothelial cells) is known to have two binding pockets: one for the Sialyl Lewis X moiety, and another for the tyrosine sulfate rich region of its native ligand PSGL-1, which is presented on leukocytes (Somers et al., Cell (2000) 103:467-79). The latter region of PSGL-1 is also rich in amino acids with carboxylate side chains. Other P- or L-selectin ligands can be sulfated, sialylated mucins presented on endothelial cells or on carcinoma cells. Notably these are also molecules presenting high densities of negatively charged sulfates and carboxylates. Heparins may mimic these natural and pathological ligands by virtue of their high density of sulfates and carboxylates, i.e., presenting a similar “clustered saccharide patch”. If the heparin chain is very short (as in FOND) it can only block one site at a time, making it a very poor inhibitor (upper panel). A somewhat longer heparin chain could interact with both binding sites on P-selectin, and have some inhibitory activity (middle panel). An even longer chain could block multiple P-selectin molecules and more dramatically affect the avidity of cell-cell interactions involving P-selectin ligands (lower panel). In contrast, the Antithrombin-Factor Xa complex is a soluble one, and a single pentasaccharide (with the sequence identical to that found in FOND) is both necessary and sufficient to bind to Antithrombin and catalyze the inactivation of Xa. Increasing the length of a heparin molecule would not change the outcome, unless there was more than one Antithrombin-binding pentasaccharide in the sequence. However, unlike the case with the multivalent, multi-site binding of P-selectin with its ligands in cell:cell interactions, the effect on Antithrombin-Xa interactions would only be additive. The specificity of heparin structure for recognition by P-selectin is also not detailed in this model. However previous work by us and others (see text) indicate a continuum of binding affinities, with 6-O-sulfation being necessary.

FIG. 7 shows P- and L-selectin-ligand interactions in normal physiology and hematogenous metastasis. Heparin therapy can minimize metastasis by inhibiting the interactions between leukocytes, platelets, and endothelial cells with tumor cell and endogenous ligands.

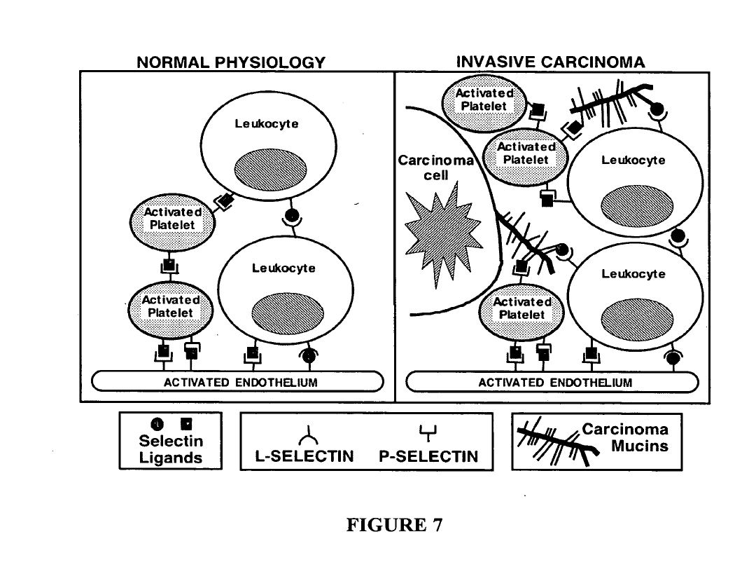

FIG. 8 shows P- and L-selectin deficiency improves long-term survival in an experimental model of hematogenous metastasis. WT and PL−/− mice were injected intravenously with MC38GFP colon carcinoma cells. Mice were monitored daily for appearance, and were euthanized when moribund to verify the presence of pulmonary metastatic foci. The number of surviving mice is plotted versus time after tumor cell injection. While all PL−/− mice appeared normal at the time of termination, 5 of 7 showed visible pulmonary metastatic foci

FIG. 9 shows that high dose heparin further improves survival in mice deficient in P- and L-selectin. PL−/− mice were injected intravenously with tumor cells at t=0, and subcutaneously with PBS or 100 U of unfractionated heparin in PBS at t=−0.5 h, +6 h, and +12 h. Mice were euthanized 50 days after injection, and the formation of pulmonary metastases was determined by quantifying the fluorescence of the lung homogenate. P-values were determined by performing a Student's T-test, assuming a two-tailed, unequal distribution.

FIGS. 10A and B demonstrate that administration of clinically relevant levels of heparin has no significant effect on formation of metastatic foci in mice deficient in both P- and L-selectin. PL−/− mice were injected intravenously with tumor cells at t=0, and subcutaneously with PBS or 19.68 U unfractionated heparin (UFH) in PBS at t=−0.5 h, +6 h, and +12 h. Mice were euthanized 55 days after injection, and the formation of pulmonary metastases determined by counting the number of visible foci (A) and by quantifying the fluorescence of the lung homogenate (B), note the split y-axis). P-values were determined as in FIG. 9.

### DETAILED DESCRIPTION

U.S. Pat. No. 6,787,365 and U.S. Pat. No. 6,596,705 are incorporated herein by reference, in their entirety, for all purposes. All patents and publications mentioned in the specification are indicative of the levels of skill of those skilled in the art to which the invention pertains. All references cited in this disclosure are incorporated by reference to the same extent as if each reference had been incorporated by reference in its entirety individually.

L-selectin, E-selectin and P-selectin mediate the initial adhesive events directing the homing of lymphocytes into lymphoid organs, as well as the interactions of leukocytes and other inflammatory cells with endothelium at sites of inflammation. L-selectin is expressed on leukocytes, E-selectin is expressed on endothelium and P-selectin is expressed on platelets and endothelium. The three selectins bind to specific carbohydrate structures on opposing cells, for example, L-selectin binds to platelets and endothelium, whereas P-selectin and E-selectin bind to leukocytes.

Selectin adhesion is involved in disorders such as pathologic reperfusion injury, inflammatory disorders and autoimmune disorders. Selectin interactions also can mediate primary adhesive mechanisms involved in the metastasis of certain epithelial cancers. Thus, selectins are potential therapeutic targets for the treatment of pathologies characterized by undesirable or abnormal interactions mediated by selectins.

In vitro and in vivo methods for identifying types and lots of heparin that inhibit P- and/or L-selectin activity are provided. Such methods provide various means for identifying forms of heparin that bind to P- and/or L-selectin. Subsequently, the identified forms of heparin may be used to inhibit metastasis of cancer cells. In addition, methods for identifying fragments of heparin that possess inhibitory activity relative to anticoagulant activity are provided.

Heparins have many other biological effects potentially relevant to solid tumor spread, including inhibition of heparanases involved in degrading basement membranes, modulatory effects on various heparin-binding growth factors or extracellular proteases, alteration of integrin functions in cell adhesion, inhibition of angiogenesis, etc. Of all these potential non-anticoagulant mechanisms, P/L-selectin inhibition is the first one likely to be relevant when tumor cells initially enter the blood stream. This effect also stands at the beginning of a cascade of events involved in survival of tumor cells, before their eventual extravasation and establishment as metastatic foci. As with any cascade, blocking the first step can make all subsequent mechanisms practically irrelevant. Indeed, it has been shown that effects of single-dose UFH given before intravenous tumor cell introduction can be explained by inhibition of P/L-selectin, since heparin had no further effects on metastasis in mice with a combined deficiency of both selectins. A similar result was seen regarding heparin effects in attenuating inflammation, with the relevant activity again limited to P- and L-selectin inhibition.

Overall, models explaining heparin action in solid tumor metastasis has been inhibition of P/L-selectins, combined with an unknown degree of blockade of intravascular fibrin formation by the fluid-phase coagulation pathway. However, the relatively high doses administered in most previous studies would be impractical to use clinically, because of excessive anticoagulation. UFH also generally has poor bioavailability, requires multiple daily dosing, and has side effects such as heparin-induced thrombocytopenia (Rosenberg R D. Semin Hematol (1997) 34 Suppl. 4:2-8; Hirsh et al., Chest (2004) 126:188 S-203S). To circumvent this, many low molecular weight heparins (LMWHs) have been created by degrading UFH using a variety of methods, including chemical depolymerization and enzymatic digestion (Rosenerg, supra; Linhardt et al., Semin Thromb Hemost (1999) 25 Suppl. 3:5-16). While LMWHs are also a mixture of fragment sizes, with molecular weight profiles ranging from 3000 to 9000 daltons, they have better kinetics and bioavailability, typically requiring only single daily doses. Taken together with a similar efficacy in clinical anticoagulation (via anti-Xa activity), and a lower incidence of side effects such as heparin-induced thrombocytopenia, they have become favored in clinical practice (Hirsh et al., supra, Valentine et al., Semin Thromb Hemost (1997) 23:173-8). Further benefits are claimed for Fondaparinux (FOND), a synthetic heparinoid pentasaccharide of defined structure that specifically binds to Antithrombin.

Heparin has been proposed to interdict metastasis during the period between initial diagnosis of early stage carcinomas and soon after their surgical removal, an idea supported by the recent finding that patients with primary tumors (but no metastases) who were treated with a LMWH had increased survival (Lee et al. J Clin Oncol (2005) 23:2123-9).

Translating all these promising ideas into clinical practice, however, requires experimental evaluation of the potential for clinically acceptable levels of the various kinds of heparins to block P/L-selectin and attenuate metastasis. However, heparin has not been used for the purpose of inhibiting L-selectin and P-selectin binding in humans because of concerns about potential undesirable side effects associated with its anticoagulant activity.

Heparin preparations that are already approved by the FDA for use as anticoagulants can be used at clinically tolerable doses (from the perspective of anticoagulation) to inhibit P- and L-selectin mediated pathologies, including ischemia, reperfusion injury, acute inflammation, chronic inflammation, and cancer metastasis. The present study provides methods for identifying types and lots of heparin preparations that mediate the activity of P- and L-selectin. Provided herein are in vivo and in vitro methods for screening heparin compositions for optimal ability to inhibit P- and L-selectin. The identified heparins can, for example, be labeled for use in the above conditions. Heparin therapy is already widely used for anticoagulant indications with manageable side effects. Also provided are heparin and heparinoid preparations useful for non-anticoagulant treatments. Also provided are fragments of heparin that have potent selectin inhibitory activity with comparison to its anticoagulant activity.

Clinical grade preparations of UFH, three types of LMWH (Tinzaparin (TINZ), Dalteparin (DALT), and Enoxaparin (ENOX)), and the synthetic pentasaccharide (FOND) are commercially available and represent the majority of heparins currently marketed for clinical use in the USA (source: Physician's Desk Reference). Clinically approved heparin formulations have widely varying abilities to inhibit P- and L-Selectin in vitro. Notably, the LMWHs are prepared by different methods of UFH degradation: TINZ, by beta-eliminative cleavage with heparinase; DALT, by deaminative cleavage with nitrous acid; and ENOX, by beta-eliminative cleavage with alkali.

The invention provides methods for identifying heparin fractions that lack substantial amounts of anticoagulant activity yet retain L-selectin and/or P-selectin inhibitory activity. The invention further provides methods of inhibiting metastasis in a subject comprising administering a heparin or heparin fraction. The invention provides methods of inhibiting L-selectin and/or P-selectin mediated metastasis in a subject by administering to the subject an amount of a fractionated heparin that does not produce substantial anticoagulant activity or undesirable bleeding in the subject. In one aspect, the concentration of heparin comprises an anti-Xa level 1 IU/ml or below. Importantly, selectin inhibition can be achieved at plasma concentrations lower than those that cause excessive anticoagulation or unwanted bleeding in a mammalian subject.

For the methods of the invention, an amount of heparin that does not produce substantial anticoagulant activity or undesirable bleeding is administered to the subject. As used herein, reference to “an amount of heparin that does not produce substantial anticoagulant activity” means an amount of heparin that does not cause bleeding complications, although a mild anticoagulant effect can occur.

Clinical signs and symptoms of undesirable bleeding include blood in the urine, or stool, heavier than normal menses, nose bleeds or excessive bleeding from minor wounds or surgical sites. Easy bruising can precede such clinical manifestations. Where undesirable bleeding occurs, heparin activity can be neutralized by administration of protamine sulfate; however this is not true of FOND.

As disclosed herein, heparin, as formulated for clinical use, can inhibit the binding of P-selectin and L-selectin to their ligands. Such amounts and methods are also useful in inhibiting metastasis. Thus, the invention provides a means to inhibit L-selectin and P-selectin mediated binding in a subject by administering heparin in an amount that does not produce substantial anticoagulant activity or undesirable bleeding in the subject. The amount of heparin administered to a subject to inhibit L-selectin or P-selectin mediated metastasis is characterized in that it does not produce undesirable bleeding as a side effect, although it can produce mild anticoagulant activity. As a result, side effects such as bleeding complications that are associated with using heparin for anticoagulant therapy are not a concern. In one aspect, the invention demonstrates that P-selectin can be inhibited at lower concentrations of heparin than L-selectin, thus providing a means for selectively inhibiting P-selecting.

Although an amount of heparin administered to inhibit L-selectin and P-selectin mediated metastasis in a subject will depend, in part, on the individual, normal adult subjects administered heparin in amounts that result in less than 0.2 units heparin/ml of plasma generally do not exhibit undesirable bleeding. A subject treated with heparin can be monitored for undesirable bleeding using various assays well known in the art. For example, blood clotting time, active partial thromboplastin time (APTT), or anti-Xa activity can be used to determine if coagulation status is undesirably increased in a subject administered heparin. Where undesirable bleeding occurs, heparin administration is discontinued.

The amount of heparin administered depends, in part, on whether L-selectin or P-selectin mediates metastasis and, therefore, whether only P-selectin, or both L-selectin and P-selectin, are to be inhibited. For example, an amount of heparin less than that used for anticoagulant therapy can be administered to a subject for the purpose of substantially inhibiting P-selectin as compared to L-selectin. The amount of heparin administered to a subject also depends on the magnitude of the therapeutic effect desired.

The invention also provides methods of screening and identifying metastasis inhibitors that inhibit interactions between P- and/or L-selectin. The method includes providing i) a heparin preparation or heparin fraction comprising a plurality of heparin molecules, wherein the preparation is obtained from an FDA-approved heparin type and lot; ii) one or more selectins selected from the group consisting of L-selectin and P-selectin; iii) a ligand for one or more of the selectins; and b) contacting a) i) with a) ii) and a) iii), simultaneously or consecutively, under conditions suitable for selectin binding to a selectin ligand; and c) detecting a reduced level of binding of the one or more selectins to a ligand in the presence of the heparin preparation compared to in the absence of the heparin preparation, wherein a reduction in binding is indicative of a composition for inhibition metastasis. Ligands useful in various methods provided herein include, but are not limited to, PSGL-1 or sialyl-Lewis^{x} (SLe^{x}). The ligand may be immobilized. The ligand may be present on a cell, such as, for example, an endothelial cell. Exemplary cells include LS180 cells.

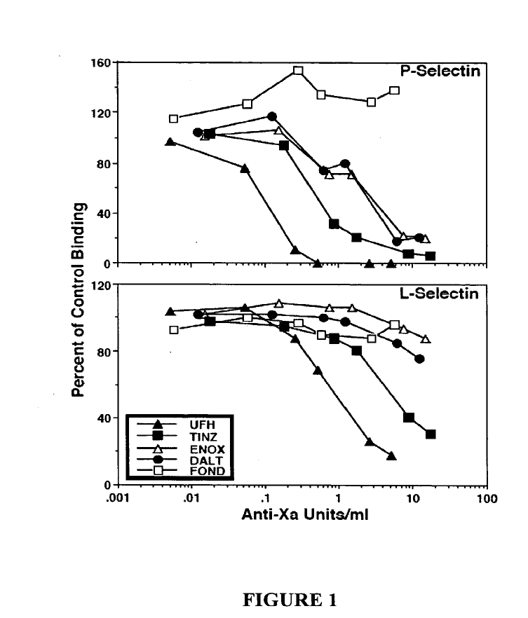

For example, P- or L-selectin chimeras are immobilized on Protein-A coated plates and fluorescently-labeled tumor cells are allowed to bind in the presence of varying amounts of heparins. Using this method, the invention demonstrates that when normalized to anti-Factor-Xa activity (a predictor of in vivo anticoagulant activity), UFH was the best inhibitor of both selectins (FIG. 1). Much variation was observed amongst the three LMWHs, with TINZ having higher inhibitory activity than DALT and ENOX. Interestingly, FOND, while synthetically designed specifically for its potent anticoagulant activity, had no ability to inhibit either P- or L-selectin. DALT and ENOX were capable of inhibiting P-selectin binding at higher anti-Xa concentrations (FIG. 1, top panel), but had only minimal ability to inhibit L-selectin binding (FIG. 1, bottom panel). While inhibition of P-selectin was obtained at lower relative doses than L-selectin, the overall rank order of inhibition (UFH>TINZ>DALT=ENOX>>FOND) was the same.

In addition, the amount of heparin administered will depend on the individual subject because the bioavailability of heparin within subjects is known to vary. For example, heparin dosages are sometimes administered in units heparin/kg body weight. However, the dosages of heparin needed (e.g. units heparin/kg body weight) to attain specific levels of heparin in the plasma of a subject can vary among individuals because of differences in heparin bioavailability. Thus, the heparin concentration in the blood of a subject in units/ml plasma is the more reliable measure of heparin concentration. The amount of plasma heparin in a subject can be determined using titration and neutralization assays with protamine sulfate (this is not true for FOND).

Heparin, as used herein, refers to heparin, low molecular weight heparin, unfractionated heparin, heparin salts formed with metallic cations (e.g., sodium, calcium or magnesium) or organic bases (e.g., diethylamine, triethylamine, triethanolamine, etc.), heparin esters, heparin in fatty acid conjugates, heparin bile acid conjugates, and heparin sulfate.

As used herein, the term “inhibit binding” relative to the effect of a given concentration of a heparin on the binding of a P- and/or L-selectin or L-selectin to its ligand refers to a decrease in the amount of binding of the P- and/or L-selectin or L-selectin to its ligand relative to the amount of binding in the absence of heparin, and includes both a decrease in binding as well as a complete inhibition of binding.

An “effective amount” or “pharmaceutically effective amount” of heparin as provided herein is meant a nontoxic but sufficient amount of heparin to provide the desired therapeutic effect. The exact amount required will vary from subject to subject, depending on age, general condition of the subject, the severity of a cell proliferative disorder or other P- and/or L-selectin mediated disorder, and the particular heparin, heparin fraction etc. administered. An appropriate “effective” amount in any individual case may be determined by one of ordinary skill in the art by reference to the pertinent texts and literature and/or using routine experimentation.

By “pharmaceutically acceptable” is meant a carrier comprised of a material that is not biologically or otherwise undesirable. The term “carrier” is used generically to refer to any components present in the pharmaceutical formulations other than the active agent or agents, and thus includes diluents, binders, lubricants, disintegrants, fillers, coloring agents, wetting or emulsifying agents, pH buffering agents, preservatives, and the like. Delayed and sustained release delivery formulations can be formulated based upon expertise in the art.

The terms “treating” and “treatment” as used herein refer to reduction in severity and/or frequency of symptoms, elimination of symptoms and/or underlying cause, prevention of the occurrence of symptoms and/or their underlying cause, and improvement or remediation of damage. Thus, for example, the present method of “treating” metastasis or cell proliferative disorder (e.g., cancer) encompasses inhibition or reduction of tumor foci, cell proliferative capacity and the like.

The invention includes, in one aspect, administering an effective amount of heparin (e.g., heparin of a desired molecular weight) to a subject to inhibit the adhesion of metastatic cells to the endothelium.

The heparin used in the methods and compositions of the invention can be either a commercial heparin preparation of pharmaceutical quality or a crude heparin preparation, such as is obtained upon extracting active heparin from mammalian tissues or organs. The commercial product (USP heparin) is available from several sources (e.g., SIGMA Chemical Co., St. Louis, Mo.), generally as an alkali metal or alkaline earth salt (most commonly as sodium heparin). Alternatively, the heparin can be extracted from mammalian tissues or organs, particularly from intestinal mucosa or lung from, for example, beef, porcine and sheep, using a variety of methods known to those skilled in the art (see, e.g., Coyne, Erwin, Chemistry and Biology of Heparin, (Lundblad, R. L., et al. (Eds.), pp. 9-17, Elsevier/North-Holland, N.Y. (1981)).

Heparin and heparin-like compounds have also been found in plant tissue where the heparin or heparin-like compound is bound to the plant proteins in the form of a complex. Heparin and heparin-like compound derived from plant tissue are of particular importance because they are considerably less expensive than heparin and heparin-like compounds harvested from animal tissue.

Plants which contain heparin or heparin-like compounds such as physiologically acceptable salts of heparin, or functional analogs thereof may also be a suitable source for the invention. Typical plant sources of heparin or heparin-like compounds include artemisia princeps, nothogenia fastigia (red seaweed), copallina pililifera (red algae), cladophora sacrlis (green seaweed), chaetomorpha anteninna (green seaweed), aopallina officinalis (red seaweed), monostrom nitidum, laminaria japonica, filipendula ulmaria (meadowsweet), ecklonia kuroma (brown seaweed), ascophyllum nodosum (brown seaweed), ginkgo biloba, ulva rigida (green algae), stichopus japonicus (seacucumber), panax ginseng, spiralina maxima, spirulina platensis, laurencia gemmifera (red seaweed), and larix (larchwood).

The heparin may be low molecular weight heparin (LMWH) or, alternatively, standard or unfractionated heparin. LMWH, as used herein, includes reference to a heparin preparation having an average molecular weight of about 3,000 Daltons to about 10,000 Daltons, Typically about 4,000 Daltons to about 8,000 Daltons. LMWH may include saccharides in smaller percentages that exceed the upper end of the range. For example, tinzaparin includes a minor amount of heparin saccharides that are larger than 8,000 daltons. Such LMWHs are commercially available from a number of different sources. The heparin compounds of the invention can be prepared using a number of different separation or fractionation techniques known to and used by those of skill in the art. Such techniques include, for example, gel permeation chromatography (GPC), high-performance liquid chromatography (HPLC), ultrafiltration, size exclusion chromatography, and the like.

LMWHs are currently produced in several different ways: (i) enrichment of LMWH present in standard heparin by fractionation; ethanol and or molecular sieving e.g., gel filtration or membrane filtration; (ii) controlled chemical depolymerization (by nitrous acid, beta-elimination or periodate oxidation); and (iii) enzymatic depolymerization by heparinases. The conditions for depolymerization can be carefully controlled to yield products of desired molecular weights. Nitrous acid depolymerization is commonly used. Also employed is depolymerization of the benzylic ester of heparin by beta-elimination, which yields the same type of fragment as enzymatic depolymerization using heparinases.

LMWHs with low anticoagulant activity and retaining basic structure can be prepared by depolymerization using periodate oxidation. Several LMWHs are available commercially: (i) Fragmin with molecular weight of 4000-6000 Daltons is produced by controlled nitrous acid depolymerization of sodium heparin from porcine intestinal mucosa by Kabi Pharmacia Sweden (see also U.S. Pat. No. 5,686,431); (ii) Fraxiparin and Fraxiparine with an average molecular weight of 4,500 Daltons are produced by fractionation or controlled nitrous acid depolymerzation, respectively, of calcium heparin from porcine intestinal mucosa by Sanofi (Chaoy laboratories); (iii) Lovenox (Enoxaparin and Enoxaparine) is produced by depolymerization of sodium heparin from porcine intestinal mucosa using beta-elimination by Farmuka S F France and distributed by Aventis under the trade names Clexane and Lovenox; and (iv) Logiparin (LHN-1, Novo, Denmark) with a molecular weight of 600 to 20,000 Daltons and with more than 70% between 1500 and 10,000 Daltons is produced by enzymatic depolymerization of heparin from intestinal mucosa, using heparinase. Exemplary low molecular weight heparin fragments include, but are not limited to, enoxaparin, dalteparin, danaproid, gammaparin, nadroparin, ardeparin, tinzaparin, certoparin and reviparin.

In another embodiment, the heparin compounds of the invention can be obtained from unfractionated heparin by first depolymerizing the unfractionated heparin to yield low molecular weight heparin and then isolating or separating out the fraction of interest. Unfractionated heparin is a mixture of polysaccharide chains composed of repeating disaccharides made up of a uronic acid residue (D-glucuronic acid or L-iduronic acid) and a D-glucosamine acid residue. Many of these disaccharides are sulfated on the uronic acid residues and/or the glucosamine residue. Generally, unfractionated heparin has an average molecular weight ranging from about 6,000 Daltons to 40,000 Daltons, depending on the source of the heparin and the methods used to isolate it.

In one embodiment, the heparin retains an ability to bind P- and/or L-selectin, but is a non-anticoagulant form. For example, heparin according to this embodiment include heparin formed by desulfating heparin at the 2-O position of uronic acid residues and/or the 3-O position of glucosamine residues of heparin. Heparin and heparan sulfate consist of repeating disaccharide units containing D-glucuronic acid (GIcA) or L-iduronic acid (IdoA) and a glucosamine residue that is either N-sulfated (GIcNS), N-acetylated (GIcNAc), or, occasionally, unsubstituted (GIcNH2) (Esko, J. D., and Lindahl, U. 2001. Molecular diversity of heparan sulfate. J. Clin. Invest. 108:169-173). The disaccharides may be further sulfated at C6 or C3 of the glucosamine residues and C2 of the uronic acid residues. The potent anticoagulant activity of heparin may depend on a specific arrangement of sulfated sugar units and uronic acid epimers, which form a binding site for antithrombin. See, e.g., Wang, L. et al. (2002) J Clin Invest, July 2002, Volume 110, Number 1, 127-136. 2-O, 3-O-desulfated heparin (2/3 DS-heparin) may be prepared according to any standard method known in the art, e.g. the method of Fryer, A. et al. (1997) Selective O-desulfation produces nonanticoagulant heparin that retains pharmological activity in the lung. J. Pharmacol. Exp. Ther. 282:208-219. The anticoagulant activity of heparin and modified heparinoids may be analyzed, e.g., by amidolytic anti-factor Xa assay as described in Buchanan, M. R., Boneu, B., Ofosu, F., and Hirsh, J. (1985) The relative importance of thrombin inhibition and factor Xa inhibition to the antithrombotic effects of heparin. Blood 65:198-201.

The heparin (e.g., a heparin fraction) alone or in combination with other P- and/or L-selectin inhibitors can inhibit interaction between P- and/or L-selectin and a ligand of P- and/or L-selectin. By inhibiting interaction is meant, e.g., that P- and/or L-selectin and its ligand are unable to properly bind to each other. Such inhibition can be the result of any one of a variety of events, including, e.g., preventing or reducing interaction between P- and/or L-selectin and the ligand, inactivating P- and/or L-selectin and/or the ligand, e.g., by cleavage or other modification, altering the affinity of P- and/or L-selectin and the ligand for each other, diluting out P- and/or L-selectin and/or the ligand, preventing surface, plasma membrane, expression of P- and/or L-selectin or reducing synthesis of P- and/or L-selectin and/or the ligand, synthesizing an abnormal P- and/or L-selectin and/or ligand, synthesizing an alternatively spliced P- and/or L-selectin and/or ligand, preventing or reducing proper conformational folding of P- and/or L-selectin and/or the ligand, modulating the binding properties of P- and/or L-selectin and/or the ligand, interfering with signals that are required to activate or deactivate P- and/or L-selectin and/or the ligand, activating or deactivating P- and/or L-selectin and/or the ligand at the wrong time, or interfering with other receptors, ligands or other molecules which are required for the normal synthesis or functioning of P- and/or L-selectin and/or its ligand.

Examples of other P- and/or L-selectin inhibitors that can be used in combination with the heparin of the invention include soluble forms of P- and/or L-selectin or the ligand, inhibitory proteins, inhibitory peptides, inhibitory carbohydrates, inhibitory glycoproteins, inhibitory glycopeptides, inhibitory sulfatides, synthetic analogs of P- and/or L-selectin or the ligand, certain substances derived from natural products, inhibitors of granular release, and inhibitors of a molecule required for the synthesis or functioning of P- and/or L-selectin or the ligand.

For example, the soluble form of either P- and/or L-selectin or the ligand, or a portion thereof, can compete with its cognate molecule for the binding site on the complementary molecule, and thereby reduce or eliminate binding between the membrane-bound P- and/or L-selectin and the cellular ligand. The soluble form can be obtained, e.g., from purification or secretion of naturally occurring P- and/or L-selectin or ligand, from recombinant P- and/or L-selectin or ligand, or from synthesized P- and/or L-selectin or ligand. Soluble forms of P- and/or L-selectin or ligand are also meant to include, e.g., truncated soluble secreted forms, proteolytic fragments, other fragments, and chimeric constructs between at least a portion of P- and/or L-selectin or ligand and other molecules. Soluble forms of P- and/or L-selectin are described in Mulligan et al., J. Immunol., 151: 6410-6417, 1993, and soluble forms of P- and/or L-selectin ligand are described in Sako et al., Cell 75(6): 1179-1186, 1993.

Inhibitory proteins that can be used in combination with a heparin of the invention include, e.g., anti-P- and/or L-selectin antibodies (Palabrica et al., Nature 359: 848-851, 1992; Mulligan et al., J. Clin. Invest. 90: 1600-1607, 1992; Weyrich et al., J. Clin. Invest. 91: 2620-2629, 1993; Winn et al., J. Clin. Invest. 92: 2042-2047, 1993); anti-P- and/or L-selectin ligand antibodies (Sako et al., Cell 75(6): 1179-1186, 1993); Fab (2) fragments of the inhibitory antibody generated through enzymatic cleavage (Palabrica et al., Nature 359: 848-851, 1992); P- and/or L-selectin-IgG chimeras (Mulligan et al., Immunol., 151: 6410-6417, 1993); and carrier proteins expressing a carbohydrate moiety recognized by P- and/or L-selectin. The antibodies can be directed against P- and/or L-selectin or the ligand, or a subunit or fragment thereof. Both polyclonal and monoclonal antibodies can be used in this invention. Typically, monoclonal antibodies are used. The antibodies have a constant region derived from a human antibody and a variable region derived from an inhibitory mouse monoclonal antibody. Antibodies to human P- and/or L-selectin are described in Palabrica et al., Nature 359: 848-851, 1992; Stone and Wagner, J. C. I., 92: 804-813, 1993; and to mouse P- and/or L-selectin are described in Mayadas et al., Cell, 74: 541-554, 1993. Antibodies to human ligand are described in Sako et al., Cell 75(6): 1179-1186, 1993. Antibodies that are commercially available against human P- and/or L-selectin include clone AC1.2 monoclonal from Becton Dickinson, San Jose, Calif.

An inhibitory peptide for use in combination with a heparin of the invention can, e.g., bind to a binding site on the P- and/or L-selectin ligand so that interaction as by binding of P- and/or L-selectin to the ligand is reduced or eliminated. The inhibitory peptide can be, e.g., the same, or a portion of, the primary binding site of P- and/or L-selectin, (Geng et al., J. Biol. Chem., 266: 22313-22318, 1991, or it can be from a different binding site. Inhibitory peptides include, peptides or fragments thereof which normally bind to P- and/or L-selectin ligand, synthetic peptides and recombinant peptides. In another embodiment, an inhibitory peptide can bind to a molecule other than P- and/or L-selectin or its ligand, and thereby interfere with the binding of P- and/or L-selectin to its ligand because the molecule is either directly or indirectly involved in effecting the synthesis and/or functioning of P- and/or L-selectin and/or its ligand.

Inhibitory carbohydrates include oligosaccharides containing sialyl-Lewis a or sialyl-Lewis x or related structures or analogs, carbohydrates containing 2,6 sialic acid, heparin fractions depleted of anti-coagulant activity, heparin oligosaccharides, e.g., heparin tetrasaccharides or low weight heparin, and other sulfated polysaccharides. Inhibitory carbohydrates are described in Nelson et al., Blood 82: 3253-3258, 1993; Mulligan et al., Nature 364: 149-151, 1993; Ball et al., J. Am. Chem. Soc. 114: 5449-5451, 1992; De Frees et al., J. Am. Chem. Soc. 115: 7549-7550, 1993. Inhibitory carbohydrates that are commercially available include, e.g., 3′-sialyl-Lewis x, 3′-sialy-Lewis a, lacto-N-fucopentose III and 3′-sialyl-3-fucosyllactose, from Oxford GlycoSystems, Rosedale, N.Y.

Inhibitory glycoproteins, e.g., PSGL-1, 160 kD monospecific P- and/or L-selectin ligand, lysosomal membrane glycoproteins, glycoprotein containing sialyl-Lewis x, and inhibitory sulfatides (Suzuki et al., Biochem. Biophys. Res. Commun. 190: 426-434, 1993; Todderud et al., J. Leuk. Biol. 52: 85-88, 1992) that inhibit P- and/or L-selectin interaction with its ligand can also be used in this invention in combination with a heparin of the invention.

Synthetic analogs or mimetics of P- and/or L-selectin or the ligand also can serve as inhibitory agents. P- and/or L-selectin analogs or mimetics are substances which resemble in shape and/or charge distribution P- and/or L-selectin. An analog of at least a portion of P- and/or L-selectin can compete with its cognate membrane-bound P- and/or L-selectin for the binding site on the ligand, and thereby reduce or eliminate binding between the membrane-bound P- and/or L-selectin and the ligand. Ligand analogs or mimetics include substances which resemble in shape and/or charge distribution the carbohydrate ligand for P- and/or L-selectin. An analog of at least a portion of the ligand can compete with its cognate cellular ligand for the binding site on the P- and/or L-selectin, and thereby reduce or eliminate binding between P- and/or L-selectin and the cellular ligand. In certain embodiments which use a ligand analog, the sialic acid of a carbohydrate ligand is replaced with a group that increases the stability of the compound yet still retains or increases its affinity for P- and/or L-selectin, e.g. a carboxyl group with an appropriate spacer. An advantage of increasing the stability is that it allows the agent to be administered orally. Sialyl-Lewis x analog with glucal in the reducing end and a bivalent sialyl-Lewis x anchored on a galactose residue via beta-1,3- and beta-1,6-linkages also inhibit P- and/or L-selectin binding (DeFrees et al., J. Am. Chem. Soc., 115: 7549-7550, 1993).

An inhibitor of granular release also interferes with P- and/or L-selectin expression on the cell surface, and therefore interferes with P- and/or L-selectin function. By granular release is meant the secretion by exocytosis of storage granules containing P- and/or L-selectin: Weibel-Palade bodies of endothelial cells or [agr]-granules of platelets. The fusion of the granular membrane with the plasma membrane results in expression of P- and/or L-selectin on the cell surface. Examples of such agents include colchicine. (Sinha and Wagner, Europ. J. Cell. Biol. 43: 377-383, 1987).

Active agents also include inhibitors of a molecule that is required for synthesis, post-translational modification, or functioning of P- and/or L-selectin and/or the ligand, or activators of a molecule that inhibits the synthesis or functioning of P- and/or L-selectin and/or the ligand. Agents include cytokines, growth factors, hormones, signaling components, kinases, phosphatases, homeobox proteins, transcription factors, translation factors and post-translation factors or enzymes. Agents are also meant to include ionizing radiation, non-ionizing radiation, ultrasound and toxic agents which can, e.g., at least partially inactivate or destroy P- and/or L-selectin and/or the ligand.

As noted above, in certain embodiments of the invention, the active agent may be monoclonal and/or polyclonal antibodies directed against P- and/or L-selectin or its ligand (e.g., PSGL-1). Mouse, or other nonhuman antibodies reactive with P- and/or L-selectin or its ligand can be obtained using a variety of immunization strategies, such as those described in U.S. Pat. Nos. 6,210,670; 6,177,547; and 5,622,701; each of which is incorporated by reference herein. In some strategies, nonhuman animals (usually nonhuman mammals), such as mice, are immunized with P- and/or L-selectin antigens. Typical immunogens are cells stably transfected with P- and/or L-selectin and expressing these molecules on their cell surface. Other immunogens include P- and/or L-selectin proteins or epitopic fragments of P- and/or L-selectin containing the segments of these molecules that bind to the exemplified reacting antibodies.

Antibody-producing cells obtained from the immunized animals are immortalized and selected for the production of an antibody which specifically binds to multiple selectins. See, Harlow & Lane, Antibodies, A Laboratory Manual (C.S.H.P. N.Y., 1988).

Other selectin inhibitors that can be used in combination with a heparin of the invention contemplated for use in the invention include heparinoids that block P- and/or L-selectin binding; the carbohydrate molecule fucoidin and synthetic sugar derivatives such as OJ-R9188 which block selectin-ligand interactions; the carbon-fucosylated derivative of glycyrrhetinic acid GM2296 and other sialyl Lewis X glycomimetic compounds; inhibitors of P- and/or L-selectin expression such as mycophenolate mofetil, the proteasome inhibitor ALLN, and antioxidants such as PDTC; sulfatide and sulfatide analogues such as BMS-190394; the 19 amino acid terminal peptide of PSGL1, other PSGL-1 peptides, PSGL-1 fusion proteins, PSGL-1 analogues, and selective inhibitors of PSGL-1 binding such as beta-C-mannosides; benzothiazole compounds derived from ZZZ21322 such as Compound 2; and/or statins, particularly Simvastatin which is marketed by Merck as Zocor.

In certain embodiments, the invention contemplates the use of enhancers, e.g. liposomes and/or nanocapsules for the delivery of a heparin of the invention alone or in combination with other inhibitors, such that the agent is complexed with an enhancer compound effective to enhance the uptake of the heparin from the gastrointestinal (GI) tract into the bloodstream. Such formulations may be used for the introduction of pharmaceutically-acceptable formulations of the heparins, antibodies, and/or other active agents disclosed herein. The formation and use of liposomes is generally known to those of skill in the art. See, e.g., Backer, M. V., et al. (2002) Bioconjug Chem 13(3):462-7.

In one embodiment, 1-(acyloxyalkyl)imidazoles (AAI) are of use in the instant invention as nontoxic, pH-sensitive liposomes. AAI are incorporated into the liposomes as described in Chen, F, et al. (2003) Cytosolic delivery of macromolecules: I. Synthesis and characterization of pH-sensitive acyloxyalkylimidazoles Biochimica et Biophysica Acta (BBA)—Biomembranes Volume 1611, Issues 1-2, pp 140-150. Exemplary 1-(acyloxyalkyl)imidazoles (AAI) may be synthesized by nucleophilic substitution of chloroalkyl esters of fatty acids with imidazole. The former may be prepared from fatty acid chloride and an aldehyde. When incorporated into liposomes, these lipids show an apparent pKa value ranging from 5.12 for 1-(palmitoyloxymethyl)imidazole (PMI) to 5.29 for 1-[(alpha-myristoyloxy)ethyl]imidazole (alpha-MEI) as determined by a fluorescence assay. When the imidazole moiety is protonated, the lipids are surface-active, as demonstrated by hemolytic activity towards red blood cells. AAI may be hydrolyzed in serum as well as in cell homogenate. They are significantly less toxic than biochemically stable N-dodecylimidazole (NDI) towards Chinese hamster ovary (CHO) and RAW 264.7 (RAW) cells as determined by MTT assay.

A number of absorption enhancers are known in the art and may be utilized in the invention. For instance, medium chain glycerides have demonstrated the ability to enhance the absorption of hydrophilic drugs across the intestinal mucosa (Pharm. Res. Vol 11:1148-54 (1994)). Sodium caprate has been reported to enhance intestinal and colonic drug absorption by the paracellular route (Pharm. Res. 10:857-864 (1993); Pharm. Res. 5:341-346 (1988)). U.S. Pat. No. 4,545,161 discloses a process for increasing the enteral absorbability of heparin and heparinoids by adding non-ionic surfactants such as those that can be prepared by reacting ethylene oxide with a fatty acid, a fatty alcohol, an alkylphenol or a sorbitan or glycerol fatty acid ester.

A method for enhancing heparin absorption through mucous membranes by co-administering a sulfone and a fatty alcohol along with the heparin can be used (U.S. Pat. No. 3,510,561). U.S. Pat. No. 4,239,754 to Sache et al. describes liposomal formulations for the oral administration of heparin, intended to provide for a prolonged duration of action. The heparin is retained within or on liposomes, which are typically formed from phospholipids containing acyl chains deriving from unsaturated fatty acids.

Other delivery methods for heparin of the invention are described in U.S. Pat. No. 4,654,327 to Teng (pertains to the oral administration of heparin in the form of a complex with a quaternary ammonium ion), U.S. Pat. No. 4,656,161 to Herr (describes a method for increasing the enteral absorbability of heparin or heparinoids by orally administering the drug along with a non-ionic surfactant such as polyoxyethylene-20 cetyl ether, polyoxyethylene-20 stearate, other polyoxyethylene (polyethylene glycol)-based surfactants, polyoxypropylene-1 5 stearyl ether, sucrose palmitate stearate, or octyl-beta-D-glucopyranoside), U.S. Pat. No. 4,695,450 to Bauer describes an anhydrous emulsion of a hydrophilic liquid containing polyethylene glycol, a dihydric alcohol such as propylene glycol, or a trihydric alcohol such as glycerol, and a hydrophobic liquid, particularly an animal oil, a mineral oil, or a synthetic oil), U.S. Pat. No. 4,703,042 to Bodor describes oral administration of a salt of polyanionic heparinic acid and a polycationic species), U.S. Pat. No. 4,994,439 to Longenecker et al. describes a method for improving the transmembrane absorbability of macromolecular drugs such as peptides and proteins, by co-administering the drug along with a combination of a bile salt or fusidate or derivative thereof and a non-ionic detergent (surfactant)), U.S. Pat. No. 5,688,761 to Owen et al. (focuses primarily on the delivery of peptide drugs using a water-in-oil microemulsion formulation that readily converts to an oil-in-water emulsion by the addition of an aqueous fluid, whereby the peptide or other water-soluble drug is released for absorption by the body), U.S. Pat. Nos. 5,444,041, 5,646,109 and 5,633,226 to Owen et al. (directed to water-in-oil microemulsions for delivering biologically active agents such as proteins or peptides, wherein the active agent is initially stored in the internal water phase of the emulsion, but is released when the composition converts to an oil-in-water emulsion upon mixing with bodily fluids), U.S. Pat. No. 5,714,477 to Einarsson (describes a method for improving the bioavailability of heparin, heparin fragments or their derivatives by administering the active agent in combination with one or several glycerol esters of fatty acids), U.S. Pat. No. 5,853,749 to New (describes a formulation for buffering the gut to a pH in the range of 7.5 to 9 by coadministering a biologically active agent with a bile acid or salt and a buffering agent).

In one embodiment, the present dosage forms are delayed release in nature, such that the release of composition from the dosage form is delayed after oral administration, and typically will occur in the lower GI tract. After reaching the intended release site, there may or may not be a further mechanism controlling release of the composition from the dosage form. That is, delayed release of the composition from the dosage form may be immediate and substantially complete at the intended release site, or, alternatively, release at the intended site may occur in a sustained fashion over an extended period of time, or in a staged or pulsatile fashion. For example, heparin can be delivered by external internal implantable pumps. Such pumps can deliver basal and/or bolus amounts of heparin.

As described above, a heparin of the invention alone or in combination with additional selectin inhibitors is administered in an amount effective to inhibit binding of metastatic cancer cells to P- and/or L-selectin. This binding inhibition may be assayed by a number of methods known in the art.

The heparin of the invention alone or in combination with other selectin inhibitors can be incorporated into a variety of formulations for therapeutic administration. More particularly, the heparin alone or in combination with other agents can be formulated into pharmaceutical compositions by combination with appropriate, pharmaceutically acceptable carriers or diluents, and may be formulated into various preparations, including in liquid forms, such as slurries, and solutions. Administration of the active agent can be achieved by oral administration.

Suitable formulations for use in the invention may be found in Remington's Pharmaceutical Sciences (Mack Publishing Company, Philadelphia, Pa., 19th ed. (1995)), the teachings of which are incorporated herein by reference. Moreover, for a brief review of methods for drug delivery, see, Langer, et al (1990) Science 249:1527-1533, the teachings of which are incorporated herein by reference. The pharmaceutical compositions described herein can be manufactured in a manner that is known to those of skill in the art, i.e., by means of conventional mixing, dissolving, levigating, emulsifying, entrapping or lyophilizing processes. The following methods and excipients are merely exemplary and are in no way limiting.

Pharmaceutical compositions suitable for use in the invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein.

The pharmaceutical compositions of the invention may be manufactured using any conventional method, e.g., mixing, dissolving, granulating, levigating, emulsifying, encapsulating, entrapping, melt-spinning, spray-drying, or lyophilizing processes. However, the optimal pharmaceutical formulation will be determined by one of skill in the art depending on the route of administration and the desired dosage. Such formulations may influence the physical state, stability, rate of in vivo release, and rate of in vivo clearance of the administered agent. Depending on the condition being treated, these pharmaceutical compositions may be formulated and administered systemically or locally.

The pharmaceutical compositions of the invention can also be administered by a number of routes, including without limitation, topically, rectally, orally, vaginally, nasally, transdermally. Enteral administration modalities include, for example, oral (including buccal and sublingual) and rectal administration. Transepithelial administration modalities include, for example, transmucosal administration and transdermal administration. Transmucosal administration includes, for example, enteral administration as well as nasal, inhalation, and deep lung administration; vaginal administration; and rectal administration. Transdermal administration includes passive or active transdermal or transcutaneous modalities, including, for example, patches and iontophoresis devices, as well as topical application of pastes, salves, or ointments.

The pharmaceutical compositions are formulated to contain suitable pharmaceutically acceptable carriers, and may optionally comprise excipients and auxiliaries that facilitate processing of the active compounds into preparations that can be used pharmaceutically. The administration modality will generally determine the nature of the carrier. For tissue or cellular administration, penetrants appropriate to the particular barrier to be permeated are used in the formulation. Such penetrants are generally known in the art. For certain preparations the formulation may include stabilizing materials, such as polyols (e.g., sucrose) and/or surfactants (e.g., nonionic surfactants), and the like.

These preparations may contain one or excipients, which include, without limitation: a) diluents such as sugars, including lactose, dextrose, sucrose, mannitol, or sorbitol; b) binders such as magnesium aluminum silicate, starch from corn, wheat, rice, potato, etc.; c) cellulose materials such as methyl cellulose, hydroxypropylmethyl cellulose, and sodium carboxymethyl cellulose, polyvinyl pyrrolidone, gums such as gum arabic and gum tragacanth, and proteins such as gelatin and collagen; d) disintegrating or solubilizing agents such as cross-linked polyvinyl pyrrolidone, starches, agar, alginic acid or a salt thereof such as sodium alginate, or effervescent compositions; e) lubricants such as silica, talc, stearic acid or its magnesium or calcium salt, and polyethylene glycol; f) flavorants, and sweeteners; g) colorants or pigments, e.g., to identify the product or to characterize the quantity (dosage) of active agent; and h) other ingredients such as preservatives, stabilizers, swelling agents, emulsifying agents, solution promoters, salts for regulating osmotic pressure, and buffers.

The pharmaceutical composition may be provided as a salt of the active agent, which can be formed with many acids, including but not limited to hydrochloric, sulfuric, acetic, lactic, tartaric, malic, succinic, etc. Salts tend to be more soluble in aqueous or other protonic solvents that are the corresponding free base forms.

As noted above, the characteristics of the agent itself and the formulation of the agent can influence the physical state, stability, rate of in vivo release, and rate of in vivo clearance of the administered agent. Such pharmacokinetic and pharmacodynamic information can be collected through pre-clinical in vitro and in vivo studies, later confirmed in humans during the course of clinical trials. Thus, for any compound used in the method of the invention, a therapeutically effective dose in mammals, particularly humans, can be estimated initially from biochemical and/or cell-based assays. Then, dosage can be formulated in animal models to achieve a desirable therapeutic dosage range that modulates P- and/or L-selectin binding.

Toxicity and therapeutic efficacy of such compounds can be determined by standard pharmaceutical procedures in cell cultures such as in vitro human umbilical vein endothelial cells or experimental animals, e.g., for determining the LD50 (the dose lethal to 50% of the population) and the ED50 (the dose therapeutically effective in 50% of the population).

For the method of the invention, any effective administration regimen regulating the timing and sequence of doses may be used. Doses of the active agent include pharmaceutical dosage units comprising an effective amount of the agent.

Typically, the active product, e.g., the heparin, will be present in the pharmaceutical composition at a concentration ranging from about 1 mg per dose to 3,000 mg per dose and, more typically, at a concentration ranging from about 40 mg (10,000 units) per dose to about 2,700 mg (300,000 units) per dose, or about 50 mg per dose to about 600 mg per dose. However, depending upon the heparin formulation (e.g. if it was a fraction that had concentration selectin inhibition, one could give less heparin). In one embodiment, the active agent is administered in a tablet or capsule designed to increase the absorption from the GI tract. In another embodiment, the active agent is contained in a solid or capsule form suitable for oral administration in total dosages between about 50 mg to about 500 mg, and typically in total dosages of 50 mg (6,250 units), 100 mg (12,500 units), 250 mg (31,250 units) or 500 mg (62,500 units).

Daily dosages may vary widely, depending on the specific activity of the particular active agent. Depending on the route of administration, a suitable dose may be calculated according to body weight, body surface area, or organ size. The final dosage regimen will be determined by the attending physician in view of good medical practice, considering various factors that modify the action of drugs, e.g., the agent's specific activity, the severity of the disease state, the responsiveness of the patient, the age, condition, body weight, sex, and diet of the patient, the severity of any infection, and the like. Additional factors that may be taken into account include time and frequency of administration, drug combination(s), reaction sensitivities, and tolerance/response to therapy. Further refinement of the dosage appropriate for treatment involving any of the formulations mentioned herein is done routinely by the skilled practitioner without undue experimentation, especially in light of the dosage information and assays disclosed, as well as the pharmacokinetic data observed in clinical trials. Appropriate dosages may be ascertained through use of established assays for determining concentration of the agent in a body fluid or other sample together with dose response data.

The frequency of dosing will depend on the pharmacokinetic parameters of the agent and the route of administration. Dosage and administration are adjusted to provide sufficient levels of the active agent or to maintain the desired effect. Accordingly, the pharmaceutical compositions can be administered in a single dose, multiple discrete doses, continuous infusion, sustained release depots, or combinations thereof, as required to maintain desired minimum level of the agent.

Short-acting pharmaceutical compositions (i.e., short half-life) can be administered once a day or more than once a day (e.g., two, three, or four times a day). Long acting pharmaceutical compositions might be administered every 3 to 4 days, every week, or once every two weeks.

Compositions comprising an active agent of the invention formulated in a pharmaceutical acceptable carrier may be prepared, placed in an appropriate container, and labeled for treatment of an indicated condition. Conditions indicated on the label may include, but are not limited to, treatment of cellular proliferative disorders and metastasis. Kits are also contemplated, wherein the kit comprises a dosage form of a pharmaceutical composition and a package insert containing instructions for use of the composition in treatment of a medical condition.

Generally, the active agents used in the invention are administered to a subject in an effective amount. Generally, an effective amount is an amount effective to (1) reduce the symptoms of the disease sought to be treated, (2) induce a pharmacological change relevant to treating the disease sought to be treated, and/or (3) prevent the symptoms of the disease sought to be treated.

The results disclosed herein, along with the established record of heparin as a therapeutic agent, indicate that heparin can be useful for inhibiting P- and/or L-selectin based interactions using amounts lower than those required for anticoagulant therapy. In particular, the invention provides a fraction of heparin comprising the higher molecular weight heparin found in tinzaparin (e.g., greater than 8,000 daltons). Ideally, the heparin fraction is greater than 8,000 daltons, but not so large as to cause undesirable side effects or reduced bioavailability. Thus, the invention provides a method of inhibiting P- and/or L-selectin binding in a subject, by administering to the subject an amount of heparin that does not produce substantial anticoagulant activity or undesirable bleeding in the subject. Further provided are methods of treating an P- and/or L-selectin related pathology by administering to a subject having the pathology an amount of heparin that does not produce substantial anticoagulant activity or undesirable bleeding in the subject.

Particular acute and chronic conditions, in which P- and/or L-selectin have a pathophysiological role can be treated using a method of the invention. For example, undesirable immune responses in which the homing or adhesion of leukocytes, neutrophils, macrophages, eosinophils or other immune cells mediated by the interaction of L-selectin with endothelial cell ligands, can be inhibited by administering heparin to the subject according to a method of the invention. Inhibition of neutrophil adherence, for example, can interrupt the cascade of damage initiated by free oxygen radical secretion and related activities that result in tissue damage and loss of myocardial contractile function present in myocardial infarction. Similarly, P- and/or L-selectin mediated adhesion of cells such as neutrophils and platelets can be inhibited in a subject if this activity is undesirable. Thus, the severity of chronic immune disorders or acute inflammatory responses can be reduced using a method of the invention.

When administered to a subject, heparin is administered as a pharmaceutical composition. Such pharmaceutical compositions of heparin are commercially available and protocols for heparin administration are well known in the art. Such compositions and administration protocols can be conveniently employed in practicing the invention. One skilled in the art would know that the choice of the particular heparin pharmaceutical composition, depends, for example, on the route of administration and that a pharmaceutical composition of heparin can be administered to a subject by various routes, including, for example, parenterally, particularly intravenously. The heparin composition can be administered by intravenous or subcutaneous injection, and administration can be as a bolus or by continuous infusion. In addition, mucosally absorbable forms of heparin can be administered orally, rectally or by inhalation, provided the amount of heparin attained in the blood does not exceed a concentration of that produces substantial anticoagulant activity or undesirable bleeding in the subject.

Although the invention has been generally described above, further aspects of the invention will be apparent from the specific disclosure that follows, which is exemplary and not limiting.

### EXAMPLES

### Example 1

Materials: The following materials were from the UCSD Medical Center Pharmacy: Unfractionated Heparin sodium (UFH) from American Pharmaceutical Partners (20,000 U/ml; lot numbers 302523, 333246); Innohep (Tinzaparin, TINZ) from Pharmion, USA (licensed from LEO Pharmaceutical Products, Denmark, 20,000 IU/ml; lot numbers E9867A and G3371A); Fragmin (Dalteparin, DALT) from Pharmacia (5000 IU/0.2 ml; lot numbers 94250A51, 94683A02, and 94802A01); Lovenox (Enoxaparin, ENOX) from Aventis (30 mg/0.3 ml; lot numbers 30324, 9367, and 9369); and, Arixtra (Fondaparinux, FOND) from Sanofi-Synthelabo (2.5 mg/0.5 ml; lot numbers 0010000003 and 0170000010). Unless otherwise noted, all remaining chemicals were purchased from Sigma Chemical Company, St. Louis.

Cell Lines: LS180 human colonic adenocarcinoma cells and MC38GFP cells (a mouse colon carcinoma cell line stably transfected with EGFP) were cultured. Mouse melanoma B16F1 cells were cultured in DMEM with 10% FCS. All media and additives were from Gibco (Invitrogen), except for FCS from HyClone. All cells were incubated at 37° C. with 5% CO₂. Prior to use, cells were released by incubation in PBS with 2 mM EDTA at 37° C. for 5-10 minutes, and washed in PBS with Ca²⁺, Mg²⁺ and Glucose before suspending in the same buffer for intravenous injection.

Mice: C57BL/6J mice from Jackson Laboratories (Bar Harbor, Me.) were fed standard chow and water ad libitum, and maintained on a 12-hour light/dark cycle. Some mice were obtained from in-house breeding of these C57BL/6J mice. All purchased mice were allowed to acclimate in the vivarium for a minimum of one week following arrival prior to beginning experiments. All experiments were performed in AAALAC-accredited vivariums on a protocol approved by the University's IACUC.

Heparin Inhibition of LS180 Binding to Selectins: Levels of heparin were normalized based on their anti-Xa activity. Binding of cells to immobilized Human selectin-Fc chimeras was studied, except that Calcein AM-loaded LS180 cells were used. Results are expressed as percent of control binding, calculated using the formula: 100*(heparin value−EDTA value)/(buffer alone value−EDTA value). Each anti-coagulant was tested in triplicate wells at each relative concentration.

Titrating Heparin Dosage via Plasma Anti-Xa Levels: Mice were injected subcutaneously with 100 ul of UFH, TINZ, or FOND diluted in PBS, at various final concentrations. Thirty minutes later, blood was collected by cardiac puncture into 1 cc syringe containing 30 ul 10 mM EDTA. Samples were centrifuged twice at 2,000 rcf at 24° C. to collect the plasma, which was stored at −80° C. until analysis for anti-Xa activity. Human antithrombin III (3.3 ug/well) (Enzyme Research Laboratories), and human factor Xa (0.02 ug/well) (Enzyme Research Laboratories), in 155 uL 25 mM HEPES/150 mM NaCl/pH 7.5 were added to 1.25 uL plasma samples, which were then incubated with 25 ug/well of a synthetic factor Xa chromogenic substrate (Chromogenix). The reaction was stopped after 15 min by adding 50 ul/well 20% acetic acid. The resulting chromophore was measured at 405 nm. Plasma heparin levels were calculated in anti-Xa units/ml by comparing against a standard curve of heparin-spiked mouse plasma samples. Standards and samples were analyzed in triplicate. Final amounts used for “1×” dosing were 6.56 U UFH, 7.32 IU TINZ, and 0.0033 mg FOND. The “3×” dosing used three-times the amount.

Carcinoma Experimental Metastasis Assay: Mice were injected subcutaneously with 100 uL PBS or heparin in 100 uL PBS. Thirty minutes afterwards, 500,000 MC38GFP cells were injected intravenously into the lateral tail vein. Mice from each studied group were injected in alternating order, and cells were resuspended by gently flicking the tube prior to aspirating the sample for each injection. Twenty-seven days after injection, the mice were euthanized, the lungs were removed and EGFP fluorescence in lysates quantified.

Melanoma Experimental Metastasis Assay: Mice were injected with heparin and 500,000 B16F1 cells using the protocol described for the carcinoma metastasis assay. Seventeen days after injection, mice were euthanized, tracheal perfusion with 10% buffered formalin was performed, and the lungs were removed and placed into 10% buffered formalin. Lungs were allowed to fix for a minimum of twenty-four hours, removed from formalin individually and photographed using a digital camera. Lung weights were determined by removing the lungs from formalin, briefly setting them on filter paper to remove excess liquid, and then weighing them on a Sartorius analytical scale.

Heparin Disaccharide Analysis: Disaccharide analysis was performed by the UCSD Glycotechnology Core Facility. Briefly, 5 ug of each heparin were dried down, resuspended in 100 mM sodium acetate, 0.1 mM calcium acetate, pH 7.0, and incubated with 5 mU each of heparin lyases I, II, and III for 18 hours at 37° C. Samples were boiled for 2 minutes and run though a prewashed Microcon 10 filter. Samples were then dried down, resuspended in MilliQ water, and separated by HPLC on a Dionex ProPac PA1 anion exchange column using MilliQ water at pH 3.5 with a sodium chloride gradient of 50-1000 mM over 60 minutes. Post-column derivatization with fluorescence detection was achieved by mixing 2-cyanoacetamide (1%) with 250 mM NaOH in the eluent stream using an Eldex dual channel pump. The eluent was then passed through an Eppendorf TC-40 reaction coil heated to 130° C., followed by a cooling bath, and then to a Jasco fluorescence detector set at an excitation of 346 nm and an emission of 410 nm. The sensitivity of this method is ˜5 pmoles.

Heparin Sizing: A TosoHaas TSKG2000SW HPLC column was run at 0.4 ml/min in 10 mM KH₂PO₄, 0.5M NaCl, and 0.2% Zwittergent (Calbiochem). The void volume was determined using blue dextran. Cytidine Monophosphate (CMP, 0.5 ug) was spiked into all samples for use as an internal control to mark the included volume. Two different lots of each heparin were evaluated. Each sample was brought up to 10 ul total volume with MilliQ water. UV absorbance was monitored throughout the 45-minute runs at a wavelength of 206 nm. In an additional run, a larger aliquot of TINZ (19.5 ul TINZ and 2.5 ug CMP marker) was run on the same column, and 200 ul fractions were collected and evaluated for their inhibitory activity against P-selectin binding to sLe^{x} (see assay details herein). The amount of uronic acid in each fraction was quantified using a standard carbazole assay.

Assay for Inhibition of P-selectin Binding to sLex: High-binding 96-well ELISA plates were coated overnight with 2 ug/ml sLe^{x}-PAA (Gycotech, Maryland) in 50 mM carbonate buffer, pH 9.5. Plates were rinsed twice with a 1:5 dilution of HPLC running buffer (final concentration of 2 mM KH₂PO₄, 0.1M NaCl, and 0.04% Zwittergent), and then blocked for 1 hour in a 1:5 dilution of HPLC running buffer+0.5% BSA. Human P-selectin chimera was pre-complexed with goat anti-human IgG-AP (BioRad) (0.25 ug:0.25 ul) in the presence of 1:5 dilutions of collected HPLC fractions (or dilutions of those fractions in column buffer) or heparin standards for one hour at room temperature with mixing. Samples were added to the blocked plate and incubated at room temperature for 2-3 hours. The plate was rinsed twice with 1:5 dilution of HPLC buffer+0.5% BSA, and then twice with a 1:5 dilution of HLPC buffer. AP substrate solution (150 ul; 10 mM p-nitrophenyl phosphate, 100 mM Na₂CO₃, 1 mM MgCl₂, pH 9.5) was added to the plate and allowed to develop at room temperature. The optical density at 405 nm was read on a SpectraMax 250 plate reader. One unit of inhibitory activity was arbitrarily defined as 1% inhibition of selectin binding, within the linear range of the assay. Results are expressed as total inhibitory units, which is calculated using the following formula: 100*[(max binding−unknown binding)/(max binding−min binding)]*(200/ul fraction tested in inhibition assay), where “max binding” is the amount of binding in the presence of a fraction that eluted prior to heparin elution and “min binding” is the amount of binding in the presence of 0.5 IU/ml TINZ.

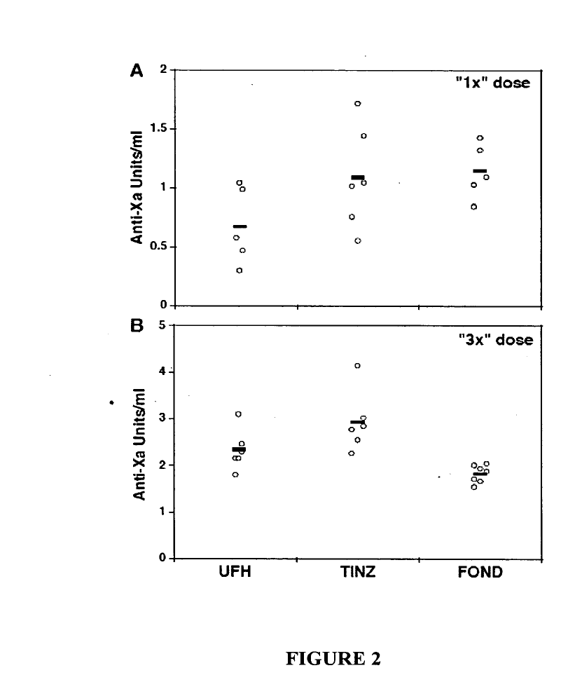

Pharmacokinetic Studies in Mice to Normalize Heparin Dosing. Additional studies comparing UFH, TINZ and FOND were performed, thus encompassing the spectrum of selectin-inhibition properties. Good documentation about the pharmacokinetics of these heparins in mice is not available in the literature. Indeed, most prior mouse studies have used high UFH doses that are likely to achieve anticoagulant effects unacceptable in human clinical use. Prior to testing these heparins in metastasis assays, studies were performed to normalize dosing, such that each administered heparin gave similar, clinically-acceptable in vivo anti-Xa levels. Subcutaneous delivery is the typical route of administration of LMWHs. Thus, subcutaneous heparin doses in mice were optimized to achieve clinically-relevant anti-Xa levels. Therapeutic levels for patients treated with heparin for venous thromboembolism are ˜1 anti-factor Xa unit/ml for LMWHs, typically measured at 3-4 hours after injection. Doses of subcutaneous heparin were systematically administered to achieve approximately similar plasma anti-Xa levels in mice. It was determined that a single dose injection amounts of “1×” UFH, TINZ, and FOND that yielded mean anti-Xa levels within this range (FIG. 2A, as in humans, there is considerable variation amongst individuals in the effects of a single subcutaneous dose). Plasma anti-Xa levels were analyzed 30 minutes after subcutaneous delivery, which is when the tumor cells would be injected into the vasculature in the planned metastasis experiments. However, pharmacokinetic studies showed that TINZ was actually cleared much faster in mice than in humans, in whom one daily dose is sufficient to maintain anti-coagulation. Single heparin doses were increased from “1×” to “3×” dosing, and analyzed the anti-Xa levels (FIG. 2B). Here, the initial peak level might be slightly higher than clinically acceptable, but practically relevant levels would be sustained a while longer. Both “1×” and “3×” heparin doses were used for the metastasis experiments, as approximating the range of concentrations that might be found in a patient on these drugs.

Carcinoma Metastasis can be Attenuated Only by Certain Heparins. All subsequent in vivo studies utilized the “experimental” model of metastasis, in which tumor cells are injected intravenously. This method provides an opportunity to study interactions between the tumor cells and blood cells within the first few hours of tumor cell entry into the vasculature, in a controlled manner at a known time point, (i.e., “spontaneous” metastasis experiments are unsuitable). Our experiments utilized a single bolus injection of heparin prior to tumor cell injection.

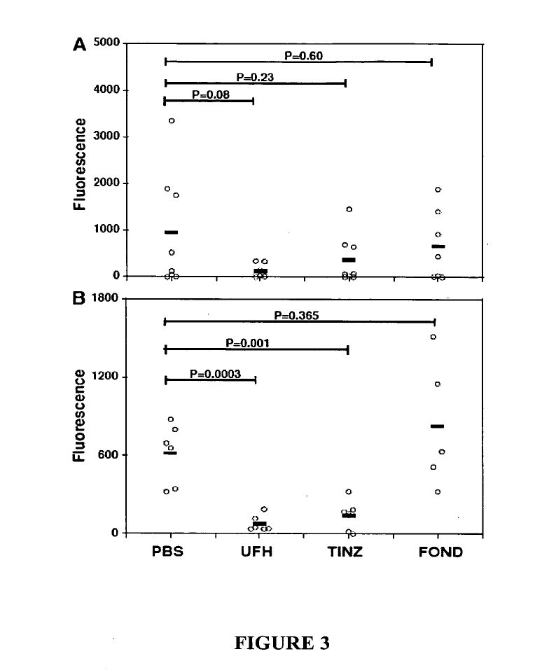

It has been demonstrated that experimental metastasis of human and mouse colon carcinoma cells could be attenuated by intravenous injection of 100 U of UFH thirty minutes prior to tumor cell injection. Studies by others using 12.5 or 60 IU of UFH prior to tumor cell injection also demonstrated a decrease in metastasis of melanoma cells. While demonstrating the potential for heparin to reduce metastasis, these and most prior studies were performed using heparin doses that are clinically unacceptable. To evaluate heparin treatment in a more clinically-relevant setting, metastasis assays were performed comparing UFH, TINZ, and FOND at “1×” and “3×” dosing. Mice were intravenously injected with syngeneic MC38GFP colon carcinoma cells known to carry selectin ligands, 30 min after subcutaneous dosing with the heparins or with a PBS control at “1×” dosing or “3×” dosing. Results with “1×” dosing demonstrated a trend in reduction of metastasis that matched the observed in vitro selectin inhibition activity (i.e., UFH>TINZ>>FOND) (FIG. 3A). However these results were not statistically significant. Injection of “3×” heparin gave almost complete attenuation of metastasis with UFH and TINZ, but still no significant difference between FOND and PBS (FIG. 3B). Notably, this dose of FOND gave plasma anti-Xa levels at or above the accepted range for clinical anticoagulation (FIG. 2B).

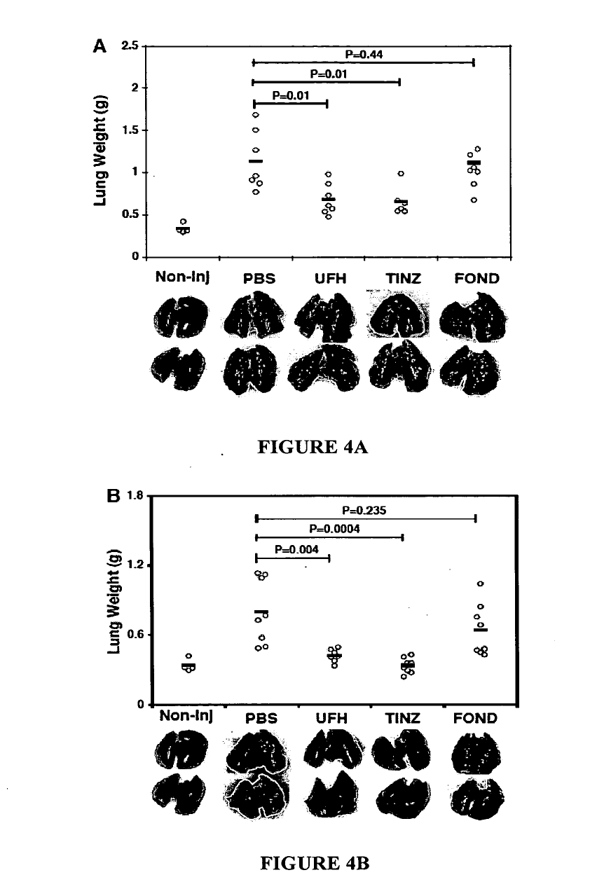

Heparin Inhibition of Melanoma Metastasis is Also Dependent on Selectin-Inhibitory Activity. The results obtained with MC38GFP cells demonstrate the relationship between inhibition of colon carcinoma metastasis and the ability of the heparin to inhibit P- and L-selectin. To determine if this phenomena was applicable to other models of cancer metastasis mice were injected intravenously with B16F1 melanoma cells thirty minutes following subcutaneous injection of “1×” dosing or “3×” dosing of UFH, TINZ, or FOND (PBS as a control). Seventeen days following injection, the lungs were excised and evaluated for the presence of metastatic foci. In lieu of counting foci, lung weights were obtained and compared to the weight of lungs from mice not injected with tumor cells. This method has been used by others, and correlated quite well with the physical appearance of the lungs (see FIG. 4). In mice that received “1×” heparin dosing, a statistically significant reduction in metastasis was observed in those that received UFH and TINZ (FIG. 4A). Again, FOND had no effect, with lungs appearing comparable to those of mice injected with PBS. When the amount of heparin was increased to “3×” dosing, an even greater reduction in metastasis was observed with UFH and TINZ treatment, with lung weights similar to those of mice that did not receive tumor cells (FIG. 4B). Again, FOND had no effect on metastasis, even at the higher dosing. Thus, a single bolus of low-dose UFH and TINZ, given just prior to injection of melanoma cells, has the ability to reduce metastasis. This trend matches that observed with colon carcinoma cells, confirming the importance of selectin inhibition (and lack of importance of anticoagulant effect) across multiple tumor cell types.

Varying Ability of LMWHs to Inhibit Selectins Does Not Correlate with Disaccharide Composition. Heparins are complex polysaccharides with a polydisperse distribution of sulfation and epimerization patterns. It has been previously shown that sulfation patterns can affect the ability of chemically-modified heparins to inhibit selectins. Given different methods of preparation of the three LMWH formulations, distinct sulfation patterns might explain their differential ability to inhibit P- and L-selectin. The structure of FOND is well known. The disaccharide composition of two lots each of UFH and of each of the three LMWHs was evaluated as described in “Materials and Methods”. No significant differences in the percentage of each disaccharide were noted. It is likely, therefore, that the differences in inhibition observed between the various LMWHs are not due to major differences in basic sulfation patterns. Rather, it would have to be due to higher-order structure and/or overall length. In support of the latter possibility, our previous work demonstrated that increasing length in the range of 1-7 disaccharides correlated with increasing ability to inhibit P- and L-selectin.

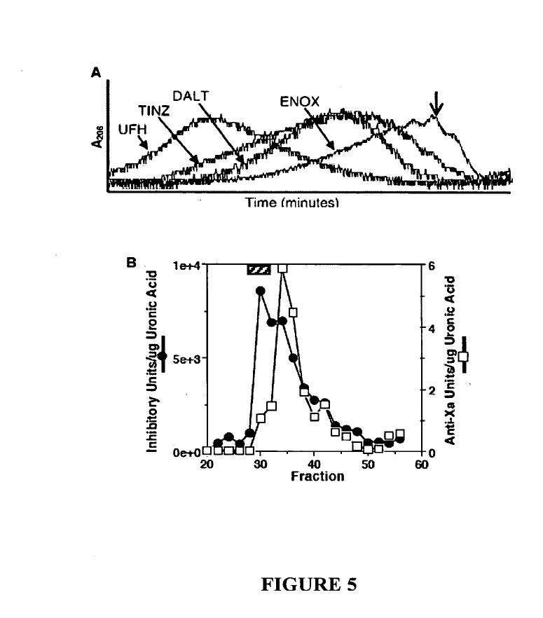

Size fractionation identifies heparins with potent selectin-inhibitory properties relative to anticoagulant activity. The package inserts that accompany the heparin formulations indicate that TINZ is likely to contain more high molecular weight (HMW) heparin fragments than either DALT or ENOX. The amount of fragments of >8000 daltons is specified as 22-36% for TINZ, 14-26% for DALT, and 0-18% for ENOX. To determine whether this potential difference in HMW content was present in our samples, size exclusion HPLC analysis was performed on all five heparins. The size profile of each heparin was determined by monitoring the UV absorbance at 206 nm (FIG. 5A). Each of the three LMWHs contained a noticeably smaller size range of heparin fragments than UFH. ENOX has a molecular weight profile lower than both TINZ and DALT. While the average molecular weight appeared to be similar for TINZ and DALT, the profile of TINZ was broader than that of DALT. Thus, TINZ contains a small amount of higher molecular weight molecules not present in DALT (FIG. 5A).

To determine if this small population of larger fragments is disproportionately involved in P-selectin inhibition, fractions were collected following HPLC size separation of a larger aliquot of TINZ. The total amount of heparin in each fraction was determined by measuring uronic acid content using a standard carbazole assay. Fractions were evaluated for their total number of P-selectin inhibitory units. A large amount of P-selectin inhibitory activity was noted in a small number of the highest molecular weight fractions (FIG. 5B). Indeed, this activity was seen even before uronic acid can be detected in the sample, indicating that a relatively small amount of HMW material has great P-selectin inhibitory activity. This result strongly supports our hypothesis that length is an important factor in determining inhibitory activity.

When evaluating the total number of anti-Xa units in the size fractionation profile of TINZ (FIG. 5B), one can see that there is a shift between the peaks of P-selectin inhibition and anti-Xa. In fact, when these variables are normalized to the amount of uronic acid in each fraction, it can be seen that there is a small subset of fractions (fractions 28-32, denoted by the hatched box at the top of the graph) that contain a very high amount of P-selectin inhibitory activity and minimal anti-Xa activity (FIG. 5B). Thus, a commercially-available heparin contains a subset of fragments that, at a given concentration, are capable of inhibiting P-selectin binding to its ligand, while only minimally affecting the coagulation process.

The close relationships of cancer and excessive systemic thrombosis are well-documented, and the need for anticoagulation in such situations is clear. Whether anticoagulation affects the spread of cancer is addressed in this disclosure. Numerous previous studies have demonstrated UFH inhibition of solid tumor metastasis in mice, and limited data suggest that the effect is likely to be relevant to humans as well. A basic assumption has therefore been that anticoagulation is the primary mechanism of its action in attenuating the metastatic process. As discussed previously, heparins are complex mixtures of bioactive molecules with many effects potentially relevant to the overall biology of solid tumors. The data indicate that the heparin effects relevant to the initial survival of tumor cells in the circulation are mainly due to inhibition of P/L-selectins, possibly along with blockade of intravascular fibrin formation via the fluid-phase coagulation pathway. Should heparin be given perioperatively as suggested, its other effects would benefit the patient during the time when tumor cells are not actively in the vasculature, as it has the potential to decrease primary tumor growth and invasion, as well as growth of established metastatic foci, due to inhibition of angiogenesis, heparanases, etc.

Almost all studies in rodents have used heparin at relatively high doses, and analysis of the various types of currently marketed heparins at clinically-relevant doses had not been performed. The present disclosure demonstrates for the first time that the ability of various heparins to inhibit P- and L-selectin in vitro correlates with their ability to inhibit metastasis of two different types of syngeneic murine tumors. This reduction of metastasis is also shown to be independent of the heparins' anticoagulant activity, since FOND, an excellent anticoagulant, had no ability to inhibit metastasis at the same level of clinically-tolerable anti-Xa activity, measured in vivo. In this regard, recent studies of metastasis inhibition with hirudin (a potent anti-thrombin) in mice used a dose far above that recommended in humans, and caused anticoagulation levels sometimes beyond the upper limits of detection of their assay. Thus, while the previously reported effects of high dose heparin and hirudin on fibrin formation supporting tumor metastasis are likely true, they may not be very relevant to the clinical situation in human patients. It was recently reported that the platelet and leukocyte-mediated P/L-selectin dependent microangiopathic coagulopathy of Trousseau Syndrome can be induced by injecting tumor mucins into mice, even in the presence of hirudin.

The data provided herein indicate that selectin inhibition is an important action of heparin affecting tumor metastasis at clinically-relevant doses. The rank order of each heparin's ability to inhibit P- and L-selectin in vitro matched the effect on metastasis attenuation in vivo. Also, these studies used single boluses of heparin yielding clinically-tolerable anti-Xa levels that are cleared from the system within a few hours. Thus, many of the other subsequent actions of heparin (e.g. angiogenesis inhibition, heparanase inhibition, etc.) are likely irrelevant to the disclosed metastasis studies, as the single-dose heparin is not in the system long enough to influence these interactions. Moreover, these other actions of heparins are downstream of the selectin effect in the described metastasis model, as tumor cells are introduced directly into the vasculature, where they interact first with P- and L-selectin bearing blood and endothelial cells. In the clinical setting of continued heparin administration, they may or may not contribute to varying extents, in different situations. It should be noted that, in the clinical setting heparin would remain in the circulation for longer following each dose (because of its increased half-life in humans). Also there would be a more extended duration of therapy. Thus, the dramatic effects seen in these single-injection studies would likely be even more pronounced in the clinical setting.

Platelets and leukocytes may support metastasis by interacting with selectin-ligands expressed on the surface of tumor cells. However, the melanoma cell line used in these studies was previously shown to express low levels of sLex, a main component of selectin ligands, and experiments performed in our laboratory indicated that recombinant P-selectin binds these cells minimally. This indicates that heparin inhibition of the melanoma cells might be due also to inhibition of endogenous selectin-ligand interactions (e.g., between PSGL-1 and P-selectin). This is supported by the recent finding that platelet aggregates around tumor cells can occur even when they do not carry P-selectin ligands. Interruption of these platelet aggregates by heparin inhibition of P-selectin and/or blockade of other effects of L-selectin may be sufficient to diminish metastasis. Therefore, heparin therapy is not necessarily limited to patients whose tumor cells carry selectin ligands.

This work provides methods for designing a prospective clinical trial evaluating pre-, peri-, and post-operative heparin therapy in relation to surgery to remove a primary malignancy, which is a period of time in which malignant cells can enter the vasculature. It also demonstrates the importance of choosing a heparin preparation known to be a potent inhibitor of P/L-selectin binding. The in vitro and in vivo data presented here would indicate that TINZ would have more of an increase in metastasis-free survival than DALT and ENOX, and that FOND would have no effect on the outcome. Therefore, recent clinical trials demonstrating an improvement in patient survival with DALT therapy might have seen an even bigger effect if TINZ had been included in their studies. Identified herein is an LMWH, which traditionally carry fewer risks for harmful side effects, that is also capable of reducing metastasis via selectin inhibition. Additionally, the present studies evaluating various fractions of TINZ show that it should eventually be possible to isolate a subset of heparin fragments that allow administration of very low doses not affecting a patient's coagulation state, but still having a significant ability to inhibit P-selectin. Finally, as anti-coagulant therapy is frequently needed in cancer patients to treat thrombosis anyhow, the present data indicates that more attention should be paid in choosing the anticoagulant, as it might be possible to improve survival in a way that is independent of anti-coagulation.

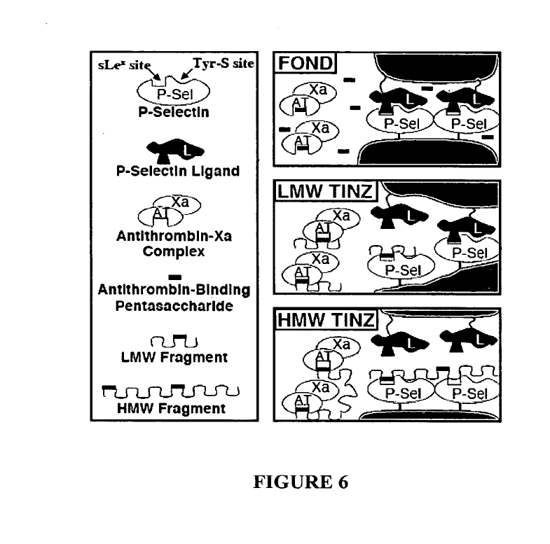

While not necessary to identify the theory of how the invention works, a discussion of the possible mechanisms of action of the disclosed heparins and heparinoids is provided. Thus, a brief discussion of the potential reasons for the differences in effects on anticoagulation and selectin inhibition is warranted (see FIG. 6 for details). The most likely reasons are due to the extended dual-site nature of the P-selectin lectin domain, and the multivalent avidity of selectin-ligand binding involving cell surfaces. This stands in contrast to heparin-antithrombin binding, which involves only one pentasaccharide binding site with a specific requirement for the precise structure found in FOND, which is also found scattered along the length of the longer heparin chains. These concepts are modeled in FIG. 6 and explained in the figure legend. Another possible (not mutually exclusive) explanation lies with the fact that as an anionic polysaccharide increases in length, many changes potentially occur in the middle of the chain, including changes in conformation and charge. Thus, extended heparin chains may have novel internal features that are preferred by P/L-selectin.

Designing new types of heparin to decrease anti-coagulant activity yet retain other activities has been previously discussed; however, such novel modified heparins will require complete pre-clinical and Phase I-III clinical testing before they can eventually be approved for use in humans. The present disclosure demonstrates that no special modification is needed, and that an effective preparation could be isolated from a subset of fragments in currently FDA-approved forms of heparin.

While this work addresses the importance of P/L-selectin inhibition by heparin in the reduction of metastasis, the findings are also of significance to the treatment of many other human diseases in which P/L-selectin have been shown to be important. These include inflammatory diseases such as allergic dermatitis, asthma, atherosclerosis, and inflammatory bowel disease; diseases in which ischemia-reperfusion injury play a critical role, such as organ transplants, myocardial dysfunction following angioplasty of blocked coronary arteries, etc. (Bevilacqua et al., Annu Rev Med (1994) 45:361-78; Lowe et al., J Clin Invest (1997) 99:822-6; and Ley K., Trends Mol Med (2003) 9:263-8); and others, such as sickle cell disease (Matsui et al., Blood (2002) 100:3790-6).

### Example 2

Cell Lines: MC38GFP cells, mouse colon carcinoma cells stably transfected with enhanced green fluorescent protein (GFP), were cultured and prepared for injection.

Mice: C57BL/6J (WT) mice were from The Jackson Laboratories (Bar Harbor, Me.) or from in-house breeding of these mice. All purchased mice were acclimatized in the vivarium for a minimum of one week following arrival, prior to beginning experiments. Mice deficient in both P- and L-selectin (PL−/−) and syngeneic for the C57BL/6J background are known in the art. All mice were fed standard chow and water ad libitum, and maintained on a 12-hour light/dark cycle. Experiments were performed in AAALAC-accredited vivariums. In keeping with IACUC recommendations, “survival” studies did not use death as an end point, but instead used euthanasia when the mice reached an obviously moribund state (mostly immobile, hunched over, breathing rapidly, and not seeking food or water).

Carcinoma Experimental Metastasis Assay: Mice were injected subcutaneously with 100 uL PBS or heparin (either 100 U or 19.68 U) in 100 uL PBS. Heparin or PBS injections were performed at t=−0.5 h, +6 h, and +12 h in relation to tumor cell injection. MC38GFP cells were injected intravenously into the lateral tail vein at t=0. WT mice were euthanized when they appeared moribund. All surviving mice were euthanized 50 or 55 days after injection. To evaluate metastasis, visible foci on excised lungs were enumerated, and/or the GFP fluorescence of lung homogenates was quantified.

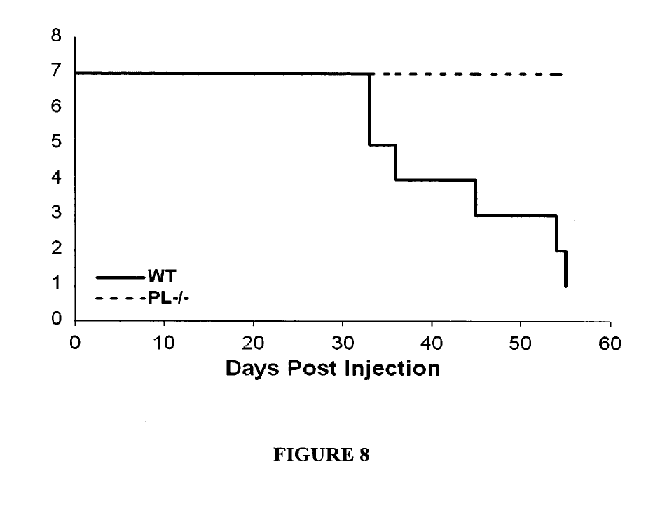

Combined deficiency of P- and L-selectin markedly extends survival of mice intravenously injected with tumor cells. Decreased formation of metastatic foci in PL−/− mice has been demonstrated in experimental metastasis studies. However, these studies were terminated at the time point when the first WT control mouse appeared moribund. Thus, the ability of P- and L-selectin deficiency to improve survival has never been evaluated. Intravenously injected WT and PL−/− mice with syngeneic mouse colon carcinoma cells were performed and the mice were monitored over a longer period of time, euthanizing individual animals only when they appeared moribund, with the typical necropsy finding being nearly complete displacement of the lung parenchyma by confluent masses of tumor cells. The first WT mice were euthanized at day 33 after tumor cell injection (FIG. 8). While the number of surviving WT mice continued to decrease over time, no PL−/− mice were observed to be moribund at the study's termination on day 55 after tumor cell injection (FIG. 8). However more than half the PL−/− mice did have visible lung metastases at day 55. Thus, while the enhanced survival with P- and L-selectin deficiency was dramatic, it would not have been absolute, if the experiment had been carried on longer. The fact that the long-living PL−/− mice still developed some metastatic lesions provided the ability to determine whether heparin would have any further effects in these animals.

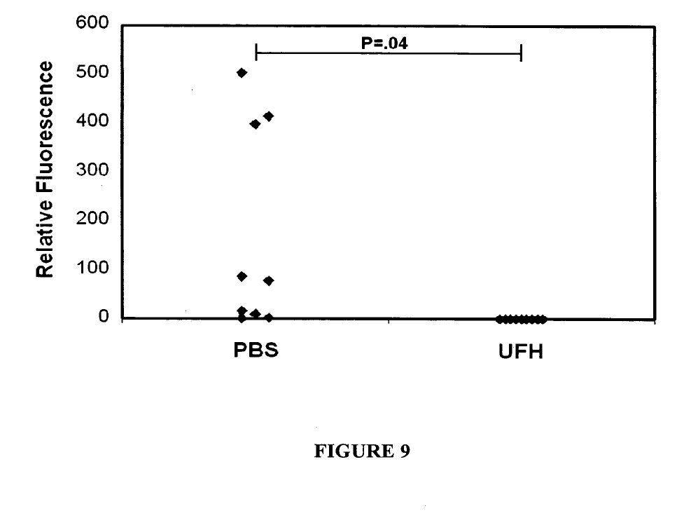

High dose heparin further reduces metastasis in P- and L-selectin deficient mice. Previous studies showed that P-selectin is likely playing a role in metastasis at very early time points in the hematogenous metastatic cascade, likely by facilitating platelet aggregation around tumor cells. Based on studies with a function blocking antibody, L-selectin also appears to be playing a relatively early role, at somewhat later time points, from ˜6-18 hours after tumor cell injection. Injection of 100 U of heparin at either 0.5 h prior to tumor cell injection or 6 h and 12 h after tumor cell injection markedly reduced formation of metastatic foci in WT mice. It is believed that the mechanism of heparin action in these studies was primarily due to its ability to inhibit P- and/or L-selectin. To pursue this hypothesis, PL−/− mice were injected with PBS or 100 U heparin at the same −0.5 h, +6 h and +12 h time points relative to injection of GFP-transfected colon carcinoma cells. These mice were kept on test for 50 days, allowing significant metastatic foci to form in at least some animals. When the GFP fluorescence of the lung homogenates was quantified as a measure of metastatic foci formation, a significant reduction was observed in the PL−/− mice that received the three heparin injections, as compared to those that received PBS control injections (FIG. 9). Thus, these high dose heparin injections have some additional effects in attenuating metastasis, which are independent of selectin inhibitory activity.

As discussed earlier, heparin has many other potential anti-metastatic effects, including anticoagulation. Some experimental studies have demonstrated that anticoagulation using the antithrombin agent hirudin can reduce metastasis. In one of these studies, 20 mg/kg of hirudin was given to mice immediately before, 4 h after, and then every other day after intravenous injection of tumor cells, for 10 days. A significant decrease in formation of metastatic foci was observed. Another group injected mice with hirudin at 10 mg/kg twenty minutes prior to tumor cell injection. Again, decreased pulmonary arrest of tumor cells with hirudin treatment was demonstrated. However, when anticoagulation by hirudin was measured at the time of tumor cell injection, the results were almost all above the limits of detection (clotting time >300 seconds in an activated partial thromboplastin time test). As this dose was about half that given in the first mentioned study, both sets of results are very likely not to be clinically relevant, given the excessive anticoagulation achieved at the doses given. Unfractionated heparin was compared with the synthetic pentasaccharide Fondaparinux, which has no selectin inhibitory activity. When given at similar clinically tolerable levels of anticoagulation, the pentasaccharide had no effect on hematogenous metastasis. The dose of 100 U heparin that is conventionally used in mouse studies achieves levels of anti-coagulation that are also unacceptable in clinical use.

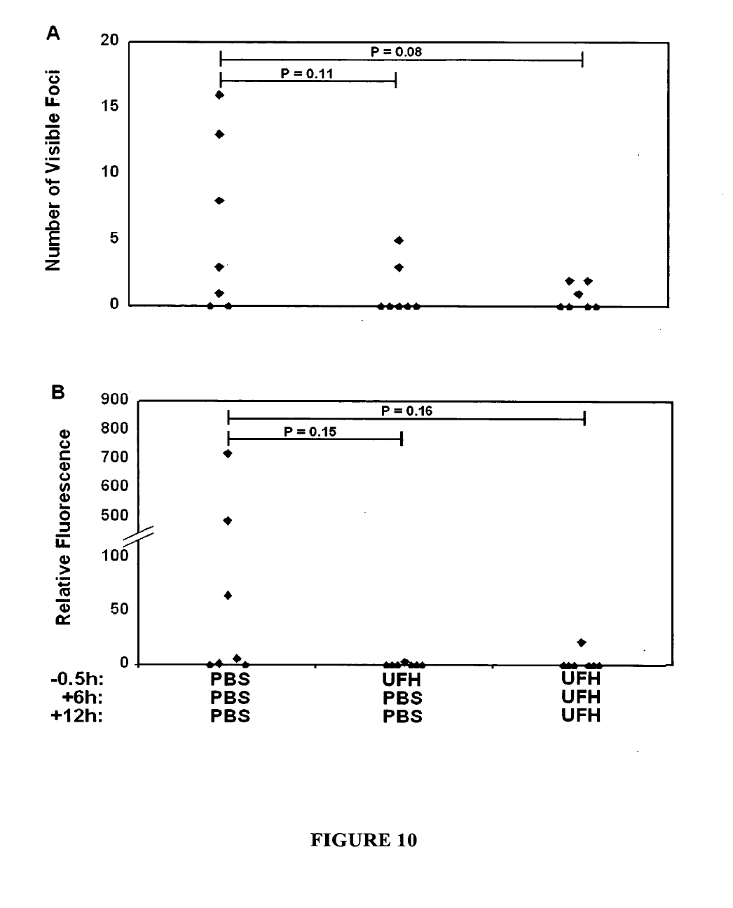

Clinically tolerable doses of heparin have no significant effect on metastasis independent of P- and L-selectin inhibition. The study examined unfractionated heparin given at clinically tolerable levels and whether the UFH has any additive effect in limiting metastasis, beyond inhibition of P- and L-selectin. Thus, a similar experiment, in which heparin was given at the same three time points, to inhibit P-selectin and L-selectin was performed. As opposed to the high dose heparin given in the previous experiment (FIG. 9), a clinically relevant dose of heparin was used. As seen by evaluating the number of visible metastatic foci (FIG. 10A) or by quantifying the fluorescence in the lung homogenate (FIG. 10B), there was no significant effect of the clinically relevant heparin injections on metastasis in the setting of P- and L-selectin deficiency (while there is a trend towards a slight improvement with heparin, this is not statistically significant, by either measure).

This dose of heparin has previously been demonstrated to have a dramatic effect on formation of metastatic foci in WT mice. Since no further effect was observed in mice deficient in both P- and L-selectin, it was concluded that clinically relevant doses of heparin attenuate metastasis mainly via inhibition of P- and L-selectin. Of course there is always a possibility that heparin also inhibits one or more additional mechanisms that are within the same linear pathway as the selectin contributions to metastasis. However, in the experimental model of metastasis, in which tumor cells are administered directly into the vasculature and immediately interact with blood cells, the selectins are likely to be involved in some of the earliest steps in the metastatic cascade. Thus, inhibiting these early steps in a cascade would render other downstream effects of heparin to be practically irrelevant. Additionally, as the doses of heparin administered in this experiment are cleared within a few hours, many of the additional effects of heparin (e.g. heparanase and angiogenesis inhibition) are likely not relevant during the time frame studied. It remains possible that heparin binding to chemokines would also be relevant during this time period.

A number of embodiments of the invention have been described. Nevertheless, it will be understood that various modifications may be made without departing from the spirit and scope of the invention. Accordingly, other embodiments are within the scope of the following claims.

1. A method for screening a composition for inhibition of selectin activity, the method comprising:
a) providing:
i) a heparin preparation comprising a plurality of heparin molecules, wherein the preparation is obtained from an FDA-approved heparin type and lot;
ii) one or more selectins selected from the group consisting of L-selectin and P-selectin;
iii) a ligand for one or more selectins selected from the group consisting of L-selectin and P-selectin; and
b) contacting a) i) with a) ii) and a) iii), simultaneously or consecutively, under conditions suitable for selectin binding to a selectin ligand; and
c) detecting a reduced level of binding of the one or more selectins to a ligand in the presence of the heparin preparation compared to in the absence of the heparin preparation, wherein a reduction in binding is indicative of a composition for inhibition of selectin activity.

1. The method of claim 1, wherein the reduced level of binding is detected using a concentration of the heparin preparation that is lower than the concentration of heparin that produces one or more activities selected from the group consisting of anticoagulant activity in vivo and undesirable bleeding in vivo.

2. The method of claim 2, wherein the concentration of the heparin preparation is lower than the concentration of heparin that produces an activity selected from the group consisting of angiogenesis inhibition, heparanase inhibition, and cytokine binding.

4. The method of claim 2, wherein the concentration of the heparin preparation does not reduce the level of binding of E-selectin to an E-selectin ligand.

5. The method of claim 3, wherein the concentration of heparin that produces the reduced level of binding of the one or more selectins to the ligand is from 2-fold to 50-fold lower than the concentration of heparin that produces excessive or dangerous anticoagulant activity in vivo.

6. The method of claim 1, wherein the ligand is PSGL-1.

7. The method of claim 1, wherein the ligand is sialyl-Lewis^{x} (SLe^{x}).

8. The method of claim 1, wherein the ligand is immobilized.

9. The method of claim 1, wherein the ligand is present on a cell.

10. The method of claim 9, wherein the cell is an endothelial cell.

11. The method of claim 9, wherein the cell is an HL-60 cell.

12. The method of claim 1, further comprising identifying the heparin preparation as therapeutic for L-selectin related pathology.

13. The method of claim 1, further comprising identifying the heparin preparation as therapeutic for P-selectin related pathology.

14. A method for screening a composition for inhibition of selectin activity, the method comprising:
a) providing:
i) a heparin preparation comprising a plurality of heparin molecules, wherein the preparation is obtained from an FDA-approved heparin lot;
ii) one or more selectins selected from the group consisting of L-selectin and P-selectin;
iii) a ligand for one or more selectins selected from the group consisting of L-selectin and P-selectin; and iii) heparin;
b) fractionating the heparin preparation of a)i) and isolating a plurality of fractions comprising heparin molecules, wherein the fractions are isolated based on the size of the heparin molecules in the fraction;
c) contacting each fraction of b) with a) ii) and a)iii), simultaneously or consecutively, under conditions suitable for selectin binding to a selectin ligand; and
d) identifying the fraction(s) that reduce the level of binding of the one or more selectins to the ligand in the presence of the fraction compared to in the absence of the fraction.

15. The method of claim 14, wherein the ligand is PSGL-1.

16. The method of claim 14, wherein the ligand is sialyl-Lewis^{x} (SLe^{x}).

17. The method of claim 14, wherein the ligand is immobilized.

18. The method of claim 14, wherein the ligand is present on a cell.

19. The method of claim 18, wherein the cell is an endothelial cell.

20. The method of claim 18, wherein the cell is an HL-60 cell.

21. A heparin fraction identified by a method comprising:
a) providing:
i) a heparin preparation comprising a plurality of heparin molecules, wherein the preparation is obtained from an FDA-approved heparin lot;
ii) one or more selectins selected from the group consisting of L-selectin and P-selectin;
iii) a ligand for one or more selectins selected from the group consisting of L-selectin and P-selectin;
b) fractionating the heparin preparation of a)i) and isolating a plurality of fractions comprising heparin molecules, wherein the fractions are isolated based on the size of the heparin molecules in the fraction;
c) contacting each fraction of b) with a) ii) and a)iii), simultaneously or consecutively, under conditions suitable for selectin binding to a selectin ligand; and
d) identifying the fraction(s) that reduce the level of binding of the one or more selectins to the ligand in the presence of the fraction compared to in the absence of the fraction.

22. The heparin fraction of claim 21, wherein the heparin comprises heparin polysaccharides of between about 8,000 and 40,000 Daltons.

23. The heparin fraction of claim 21, wherein the heparin comprises heparin polysaccharides with beta-eliminative cleavage with heparinase and a molecular weight of at least 8,000 Daltons.

24. The heparin fraction of claim 21, wherein the fraction is characterized as being the high-molecular weight fraction of tinzaparin.

25. An article of manufacture comprising packaging material and, contained within the packaging material, a heparin preparation identified by the method of claim 1, wherein the packaging material comprises a label or package insert indicating that the heparin preparation inhibits the activity of a selectin and can be used for inhibiting hematogenous metastases in a subject. The same label also provides information about the level of anticoagulant activity relative to selectin-inhibiting activity. This allows the physician to administer a dose that will provide sufficient P- and L-selectin blocking activity in vivo without causing excessive anticoagulation that might place the patient at risk of bleeding

26. The article of manufacture of claim 25, wherein the heparin preparation is a intermediate molecular weight heparin preparation comprising heparin having a molecular weight great than 8,000 daltons.

27. The article of manufacture of claim 25, wherein the heparin preparation is Tinzaparin.

28. An article of manufacture comprising packaging material and, contained within the packaging material, a heparin fraction identified by the method of claim 14, wherein the packaging material comprises a label or package insert indicating that the heparin fraction inhibits the activity of a selectin and can be used for inhibiting hematogenous metastases or any other P- and/or L-selectin mediated pathologies in a subject.

29. The article of manufacture of claim 24, wherein the selectin is selected from the group consisting of P-selectin and L-selectin.

30. A method for preventing or treating a cell proliferation disorder in a subject, the method comprising administering to the subject an effective amount of a specific inhibitor of selectin activity comprising an intermediate weight heparin, in a pharmaceutically acceptable carrier, wherein the inhibitor is a heparin preparation or a heparin fraction.

31. The method of claim 30, wherein the intermediate weight heparin comprises heparin polysaccharides of between about 8,000 and 40,000 Daltons.

32. The method of claim 30, wherein the intermediate weight heparin preparation comprises heparin polysaccharides with beta-eliminative cleavage with heparinase and a molecular weight of at least 8,000 Daltons.

33. A method for preventing or treating a cell proliferation disorder in a subject, the method comprising administering to the subject an effective amount of the heparin preparation of claim 21 in a pharmaceutically acceptable carrier.

34. A method for preventing or inhibiting metastasis or any other P- and/or L-selectin mediated pathologies in a subject, the method comprising administering to the subject an effective amount of a specific inhibitor of selectin activity, in a pharmaceutically acceptable carrier, wherein the inhibitor is a heparin preparation or a heparin fraction comprising intermediate weight heparin.

35. An article of manufacture comprising packaging material that comprises the heparin fraction of claim 21.

36. The article of manufacture of claim 35, wherein said packaging material further comprises a statement indicating that said heparin fraction inhibits the activity of a selectin.

37. The article of manufacture of claim 35, wherein said packaging material further comprises a statement indicating that said heparin fraction is useful for inhibiting hematogenous metastases.
